(19) United States
(12) Patent Application Publication
(10) Pub. No.: US 2026/0345561 A1
(43) Pub. Date: Nov. 5, 2026

(54) METHODS AND COMPOSITIONS FOR USE IN TREATING AUTOSOMAL RECESSIVE POLYCYSTIC KIDNEY DISEASE (ARPKD)

(71) Applicant: The General Hospital Corporation, Boston, MA (US)

(72) Inventors: Ken Hiratsuka, Ama-gun, Aichi (JP); Ryuji Morizane, Andover, MA (US)

(21) Appl. No.: 18/998,949

(22) PCT Filed: Jul. 26, 2023
(86) PCT No.: PCT/US2023/071029

§ 371 Date: Jan. 27, 2025

Related U.S. Application Data
(60) Provisional application No. 63/392,788, filed on Jul. 27, 2022.

(51) Int. Cl. A61K 31/423; A61K 31/192; A61K 31/407; A61K 31/506; A61P 13/12

(52) U.S. Cl. CPC A61K 31/423; A61K 31/192; A61K 31/407; A61K 31/506; A61P 13/12

(57) ABSTRACT

Provided herein are compositions and methods of use thereof for treating Autosomal recessive polycystic kidney disease (ARPKD) by targeting Rac family small GTPase 1 (RAC1) and/or Fos proto-oncogene, AP-1 transcription factor subunit (FOS) by administering a therapeutically effective amount of an inhibitor of RAC1 and/or FOS to a subject in need thereof.

### CLAIM OF PRIORITY

This application claims the benefit of U.S. Provisional Application Ser. No. 63/392,788, filed on Jul. 27, 2022. The entire contents of the foregoing are incorporated herein by reference.

### FEDERALLY SPONSORED RESEARCH OR DEVELOPMENT

This invention was made with Government support under Grant Nos. EB029388/DK133821, EB028899/DK127587, and DK129909 awarded by the National Institutes of Health. The Government has certain rights in the invention.

### SEQUENCE LISTING

This application contains a Sequence Listing that has been submitted electronically as an XML file and is hereby incorporated by reference in its entirety. Said XML file, created on Jul. 26, 2023, is named “29539-0693WO1_SL_ST26.XML” and is 12,503 bytes in size.

### TECHNICAL FIELD

Provided herein are compositions and methods of use thereof for treating Autosomal recessive polycystic kidney disease (ARPKD) by targeting Rac family small GTPase 1 (RAC1) and/or Fos proto-oncogene, AP-1 transcription factor subunit (FOS).

### BACKGROUND

Autosomal recessive polycystic kidney disease (ARPKD) is a ciliopathy that develops a severe hepatorenal disorder characterized by enlarged kidneys with progressive loss of renal function. Polycystic kidney and hepatic disease 1 (PKHD1) is a causative gene for typical cases of ARPKD, that encodes fibrocystin which localizes to the primary cilia, basal bodies, and apical membrane in kidney and liver epithelial cells (7). The reported mortality rate in neonates is as high as 30% (8, 9), and 41% of ARPKD patients who survive infancy will require renal replacement therapy by 11 years of age (10). Despite substantial progress in understanding this disease, there is no FDA-approved drug for these patients.

### SUMMARY

Provided herein are methods for treating autosomal recessive polycystic kidney disease (ARPKD). The methods comprise administering a therapeutically effective amount of an inhibitor of Rac family small GTPase 1 (RAC1) and/or an inhibitor of Fos proto-oncogene, AP-1 transcription factor subunit (FOS) to a subject in need thereof. Also provided herein are an inhibitor of Rac family small GTPase 1 (RAC1) and/or an inhibitor of Fos proto-oncogene, AP-1 transcription factor subunit (FOS) for use in a method for treating autosomal recessive polycystic kidney disease (ARPKD).

In some embodiments, the subject has been diagnosed with ARPKD. In some embodiments, the subject has a mutation in PKHD1 or has been confirmed to have a mutation in PKHD1.

In some embodiments, the treatment results in a reduction in cyst formation or growth and/or improved renal function. In some embodiments, improved renal function is determined by increased glomerular filtration rate (GFR), reduced serum creatinine level, and/or reduced Blood urea nitrogen (BUN).

In some embodiments, the inhibitor of FOS is a benzophenone derivative comprising the formula

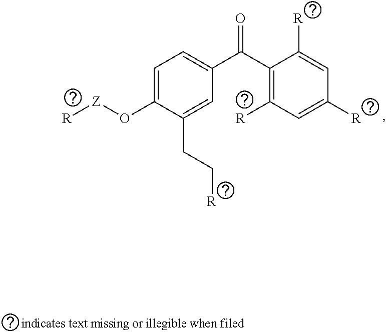

wherein
R1 represents, for example, an optionally substituted heterocyclic group, or a substituted phenyl group; Z represents, for example, an alkylene group;
R2 represents, for example, a carboxyl group optionally protected with alkyl;
R3 represents, for example, an optionally protected hydroxyl group;
R4 represents, for example, an optionally substituted cycloalkyloxy group; and
R5 represents, for example, a hydrogen atom,
or a pharmaceutically acceptable salt thereof.

In some embodiments, the inhibitor of FOS is T-5224 (3-{5-[4-(cyclopentyloxy)-2-hydroxybenzoyl]-2-[(3-hydroxy-1,2-benzisoxazol-6-yl)methoxy]phenyl}propionic acid).

In some embodiments, the inhibitor of RAC1 is an NSAID; ZINC69391 or analog 1A-116; 1D-142; anthraquinone-quinazoline hybrid 7B; NSC23766; a thioquinoline derivative such as EHT 1864; GYS32661; or MBQ-167.

In some embodiments, the NSAID is R-Naproxen or R-Ketorolac.

In some embodiments, the inhibitor of RAC1 is an inhibitory nucleic acid targeting RAC1, and/or the inhibitor of FOS is an inhibitory nucleic acid targeting FOS.

In some embodiments, the inhibitory nucleic acid targeting RAC1 or the inhibitory nucleic acid targeting FOS is an antisense RNA, antisense DNA, mixmer, gapmer, antisense oligonucleotides comprising one or more modified linkages, interference RNA (RNAi), short interfering RNA (siRNA), or a short, hairpin RNA (shRNA).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-F. CDH1⁺ distal nephron dilates in PKHD1−/− kidney organoids cultured under flow. (A) Schematic of kidney organoids-on-a-chip (flow) culture and static culture. (B) Diagram of differentiation days and culture conditions for flow and static culture. (C) Representative phase-contrast images of PKHD1−/− and PKHD1+/− organoids cultured under flow on day 16-18, day 21-25, and day 32 of differentiation. Red arrows highlight dilated tubules. Scale bars, 100 μm (upper). Representative phase-contrast images of PKHD1−/− and PKHD1+/− organoids cultured under flow on day 35 of differentiation. White arrows highlight dilated tubules. Dashed white lines highlight normal tubules. Scale bar, 50 μm (lower). (D) Immunocytochemistry with sample clearing for proximal tubule (LTL, CFTR) and distal tubule (CDH1) in PKHD1−/− organoids with and without flow on day 35. Scale bar, 100 μm. (E) Bright-field images of patient-derived ARPKD organoids with and without forskolin treatment in 3D culture on day 66. Scale bar, 250 μm (upper). Immunocytochemistry for proximal tubule (LTL), distal tubule (CDH1), and podocyte (PODXL) in PKHD1−/− organoids with and without forskolin treatment on day 35. Scale bar, 100 μm (lower). (F) Tubular diameters in nephron segments (proximal tubule (LTL) and distal tubule (CDH1)) in PKHD1−/− organoids with and without flow (left) and with and without forskolin treatment (right). Values represent mean+/−s.d. ** represents p<0.01, **** represents p<0.0001.

FIGS. 2A-L. Mechanosensing signals induced by flow. (A) Scatter plots of gene expression in PKHD1+/− organoids under flow and no flow. Red spots: higher than 2-fold. Blue spots: lower than 2-fold. Disposition of GO terms related to ciliary signals (lower). (B) Cross-sections (left and middle) and 3D rendering images (right) showing the ciliary marker TUBA1A in PKHD1−/− organoids transfected with siControl and siKIF3A under the flow condition on day 22. Scale bars, 20 μm (left and middle), 10 μm (right). (C) The percentage of ciliated cells. (D) Immunocytochemistry for LTL, CDH1, and KI67 in PKHD1−/− organoids on day 22.

Scale bars, 50 μm. (E) Tubular diameters in CDH1⁺ distal nephrons. (F) The percentage of KI67⁺ cells in CDH1⁺ distal nephrons. (G) Disposition of GO terms related to mechanosensing signals. (H) Time-lapse images of control organoids under flow. White lines: tubular basement membranes. Dashed yellow lines: the initial position of tubular basement membranes. White and red arrows: flow direction. Scale bar, 10 μm (upper). Red square: imaged area. Scale bar, 500 μm (lower left). Distance change of basement membranes from the initial position during flow (lower right). (I) Fluorescent intensity and FLIM images in a tubule with and without flow. Values of mean fluorescent intensity and fitted τ₂ lifetime are shown. Scale bars, 25 μm. (J) τ₂ lifetime change (left) and lifetime ranges (right) during perfusion and rest. (K) Fluorescence (upper left) and FLIM (lower left) images of a tubule during perfusion and rest. Histograms (right) of overall lifetimes measured in the area highlighted by red. Scale bar, 10 μm. Red and blue curves indicate lifetimes of the highest and lowest respectively during the perfusion. (L) Dextran perfusion imaging under flow. White arrow: a tubular lumen filled with dextran. Scale bar, 20 μm.

FIGS. 3A-F. Identification of novel therapeutic targets for ARPKD and drug screening tests using commercially available compounds. (A) A schematic illustrating the strategy to identify flow-specific pathways and common pathways by comparing PKHD1−/− versus PKHD1+/− organoids with flow, FK treatment, or static culture. The number of pathways is shown with parenthesis. (B) Proportions of pathways categorized in the three-level iterative analysis. (C) Schematic of the strategy to identify novel therapeutic targets for ARPKD. (D) Immunocytochemistry for proximal tubule (LTL), distal tubule (CDH1), and podocyte (PODXL) in PKHD1+/− organoids under flow and PKHD1−/− organoids treated with and without candidate compounds (T-5224, NSC23766, 2-MeOE₂, Z-DEVD-FMK, Quercetagetin, and rhCXCL16) under flow on day 35. Scale bars, 50 μm. (E) Distal tubular diameter in PKHD1+/− organoids under flow and PKHD1−/− organoids treated with and without each compound under flow on day 35. (F) A DiVenn diagram that compares pathways in our ARPKD model and human ADPKD dataset. Values represent mean+/−s.d. *** represents p<0.001 **** represents p<0.0001.

FIGS. 4A-I. Repurposing R-Naproxen, R-Ketorolac, and T-5224 as potential medications for clinical translation in ARPKD. (A) Schematic of RAC1 and FOS inhibitors. (B) Immunocytochemistry for LTL, CDH1, and KI67 in PKHD1+/− organoids under flow and PKHD1−/− organoids treated with and without candidate medications (R-Naproxen, R-Ketorolac, and T-5224) under flow on day 35. Scale bars, 50 μm. (C) Distal tubular diameter in PKHD1+/− organoids under flow and PKHD1−/− organoids treated with and without each drug under flow on day 35. (D) Whole-organoid 3D confocal imaging stacks of representative samples (PKHD1+/− organoids under flow and PKHD1−/− organoids treated with and without candidate drugs (R-Naproxen 20 μM, R-Ketorolac 1 μM and 10 μM, and T-5224) under flow) used to measure distal tubular volumes in Imaris 3D surface rendering. Scale bars, 50 μm. (E) Distal tubular volume in PKHD1+/− organoids under flow and PKHD1−/− organoids treated with and without each drug under flow on day 35. (F) Percentage of KI67⁺ cells in distal tubules in PKHD1+/− organoids under flow and PKHD1−/− organoids treated with and without each drug under flow on day 35. (G) Immunostaining of RAC1 (left), FOS (middle), and LTL (right) in human kidney samples from patients with ARPKD and normal portions of tumor nephrectomy (control). Arrowheads indicate RAC1 (left) and FOS (middle) positive cells. C; cyst, D; distal tubule, P; proximal tubule. Scale bars 25 μm (Left and middle), 50 μm (right). (H) Percentage of RAC1⁺ cells in cystic epithelial cells (ARPKD, n=4) and distal tubular cells (control, n=4). (I) Percentage of FOS' cells in cystic epithelial cells (ARPKD, n=4) and distal tubular cells (control, n=4).

FIGS. 5A-H. The mechanisms of RAC1- and FOS-mediated cystogenesis. (A) Scatter plots comparing expression profiles of PKHD1−/− and +/− organoids under flow. Red and blue dots represent >1.5-fold upregulated and downregulated genes respectively in PKHD1−/− organoids when compared to PKHD1+/− under flow conditions. (B) A schematic illustration of RAC1- and FOS-mediated signals. (C) Heat maps of microarray datasets for “negative regulation of cell population proliferation” (left) and“actin cytoskeleton” (right) in PKHD1−/− and PKHD1+/− organoids, cultured under flow, on day 35 of differentiation. (D) A schema of the culture and treatment protocol. (E) qPCR for RAC1 genes (ARFIP2 and BAIAP2) in green color and FOS genes (DUSP1, NPTX2, and PRDM1) in red color in PKHD1−/− organoids treated with R-Naproxen or T-5224 under flow conditions on day 23 of differentiation. Values are normalized against the control, PKHD1−/− organoids cultured in static conditions. (F) Immunocytochemistry for ARFIP2 and PRDM1 in PKHD1−/− organoids with and without flow, and PKHD1−/− organoids treated with R-Naproxen or T-5224 treatment under flow conditions on day 23 (left). Scale bars, 20 μm. Percentage of ARFIP2⁺CDH1⁺ tubules/CDH1⁺ tubules in PKHD1−/− organoids without flow, with flow, and with flow and R-Naproxen (upper right). Percentage of PRDM1⁺CDH1⁺ tubules/CDH1⁺ tubules in PKHD1−/− organoids without flow, with flow, and with flow and T-5224 on day 23 of differentiation (lower right). (G) Immunohistochemistry for F-actin and CDH1 in organoid tubules under static (left), flow (middle), and R-Naproxen treated flow conditions (right) on day 23. Scale bars, 20 μm. The plot profiles at the bottom show the intensity of F-actin across the tubule denoted by yellow arrows. (H) Schematic illustration of RAC1- and FOS-mediated disease mechanism. Values shown are the means+/−s.d. p-values were determined by a Student's t-test. * represents p<0.05 ** represents p<0.01.

FIGS. 6A-G. ARPKD patient-derived organoids form cysts upon forskolin treatment. (A) Exemplary diagram of differentiation days. Markers for each step of differentiation are shown; OCT4 (POU class 5 homeobox 1), SOX2 (SRY-box), SIX2 (SIX2 homeobox 2), LTL (Lotus Tetragonolobus Lectin), CDH1 (Cadherin 1), and PODXL (Podocalyxin-like protein 1). (B) Immunocytochemistry for SIX2 on day 8 in cells differentiated from H9 ESCs and patient-derived iPSCs. Scale bars, 100 μm. (C) Immunocytochemistry for proximal tubule (LTL), distal tubule (CDH1), and podocyte (PODXL) in H9-derived 2D nephron formation and patient-derived 2D nephron formation on day 28. Scale bars, 200 μm. (D) Bright-field images of patient-derived 2D nephron structures treated with and without 5 μM forskolin on day 17, 24, and 38. Red arrows indicate cystic structures. Scale bars, 1 mm. (E) Bright-field images of patient-derived kidney organoids treated with and without forskolin (5 μM, 10 μM, and 20 μM) on day 23. Scale bar, 1 mm (left), 500 μm (right). (F) Organoid diameter in patient-derived organoids treated with and without forskolin (5 μM, 10 μM, and 20 μM) on day 23. (G) Immunocytochemistry for LTL, CDH1, and KI67 on day 51 in patient-derived organoids treated with 20 μM forskolin and control organoids. Scale bars, 100 μm. Values represent mean+/−s.d. **** represents p<0.0001.

FIG. 7. Cysts in distal tubules in ARPKD sample (kidney cortex). A representative image of LTL staining in human kidney samples from ARPKD patients. Cysts are developed in LTL negative distal tubules in the kidney cortex. Asterisks indicate cysts. A scale bar: 100 μm.

FIGS. 8A-H. PKHD1−/− organoids form cysts upon forskolin treatment. (A) Deep sequence of PKHD1 gene in the mutant lines. 9 or 11 bases of nucleic acid were deleted at the exon-intron junction resulting in the translation termination soon after the mutation site. (B) Immunocytochemistry for SIX2 in PKHD1−/− and PKHD1+/− nephron progenitor cells on day 8. Scale bars, 100 μm. (C) Whole-mount immunostaining for proximal tubule (LTL), distal tubule (CDH1), and podocyte (PODXL) in PKHD1−/− and PKHD1+/− organoid on day 21. Scale bars, 100 μm. (D) Immunostaining for LTL, PKHD1 (arrows), and TUBA1A in control and PKHD1−/− organoid on day 35. Scale bar, 10 μm (upper), 20 μm (lower). (E) Immunocytochemistry for CDH1, Na⁺/K⁺ ATPase, and LTL in PKHD1+/− control and PKHD1−/− organoids with and without forskolin treatment on day 51. Scale bars, 100 μm. (F) Tubular diameter in PKHD1+/− (control) and PKHD1−/− organoids with and without forskolin (FK) 20 μM on day 51. (G) Immunostaining for K167, CDH1, and LTL in PKHD1+/− control and PKHD1−/− organoids with and without forskolin on day 35. Scale bars, 100 μm. (H) Percentage of KI67T cells in PKHD1+/− and PKHD1−/− organoids with and without forskolin 20 μM on day 35. Values represent mean+/−s.d. ** represents p<0.01, **** represents p<0.0001.

FIGS. 9A-B. Treatment of PKHD1−/− organoids with candidate compounds. (A) Schematic representation of time course to treat PKHD1−/− organoids with candidate compounds. (B) The evaluation of optimal concentration. PKHD1−/− organoids were treated with 2-MeOE₂, T-5224, Z-DEVD-FMK, Quercetagetin, NSC23766, and rhCXCL16 chemokine at various concentrations from day 16 to 35. Tubule numbers were counted on day 35. Values represent mean+/−s.d. * represents p<0.05 ** represents p<0.01 *** represents p<0.001 **** represents p<0.0001.

FIG. 10. The evaluation of optimal concentrations for R-Naproxen and R-Ketorolac treatment. PKHD1−/− organoids were treated with R-Naproxen and R-Ketorolac at various concentrations from day 16 to 35. Tubule numbers were counted on day 35. Values represent mean+/−s.d. *** represents p<0.001 **** represents p<0.0001.

FIG. 11. 3D confocal imaging of whole organoids by z-stacks from top to bottom. PKHD1−/− organoids on day 35 of culture in three different conditions: 1. No flow, 2. Flow, and 3. Flow and S-Naproxen 20 μM. Scale bars, 50 μm. A dot plot graph of tubular diameters in CDH1⁺ tubules in the PKHD1−/− organoids. Values represent mean+/−s.d. Statistical significance was attributed to values of P<0.05 as determined by one-way ANOVA with Tukey's multiple-comparisons test. **** represents p<0.0001.

FIGS. 12A-B. RAC1 and FOS expression in human kidneys and PKHD1−/− organoids. (A) Immunostaining for RAC1 in cystic epithelial cells (upper left) and proximal tubule (upper right) in human kidney samples from patients with ARPKD. Immunostaining for FOS in cystic epithelial cells (lower left) and proximal tubule (lower right) in human kidney samples from patients with ARPKD. Scale bars 25 μm. (B) Immunostaining for RAC1 in proximal tubules in PKHD1−/− with and without flow on day 35 (left). Immunostaining for LTL in proximal tubules in PKHD1−/− with and without flow on day 35 (right). Scale bars 25 μm.

FIGS. 13A-B. Expression of FOS and RAC1 in PKHD1-mutant organoids with and without flow. (A) Percentage of FOS⁺ CDH1⁺ tubules/CDH1⁺ tubules in PKHD1−/− organoids with and without flow, and in PKHD1+/− organoids with and without flow on day 35. (B) Percentage of RAC1 cells in distal nephrons in PKHD1−/− organoids with and without flow, and in PKHD1+/− organoids with and without flow on day 35. Values represent mean+/−s.d. **** represents p<0.0001.

FIG. 14. Gene expression of RAC1 and FOS downstream genes. qPCR for RAC1 genes (ARFIP2 and BAIAP2) in green color and FOS genes (DUSP1, NPTX2, and PRDM1) in red color in ARPKD patient-derived organoids treated with R-Naproxen or T-5224 under flow conditions on day 23 of differentiation. Values are normalized against the control, ARPKD patient-derived organoids cultured in static conditions. Values shown are the means+/−s.d. p-values were determined by a Student's t-test. * represents p<0.05; ** represents p<0.01 *** represents p<0.001.

FIG. 15. F-actin in PKHD1−/− organoids. Expression of F-actin and CDH1 in tubules under static, flow, and R-Naproxen treated flow conditions on day 23 of differentiation. The plot profiles of the F-actin intensity across the tubule denoted by yellow arrows.

FIGS. 16A-D. RAC1 effectors are downregulated in PKHD1−/−. A) A Venn diagram identifying 27 RAC1 effector genes altered in PKHD1−/− compared to +/− under flow. B) A bar graph showing fold changes (log 2) of the 27 RAC1 effectors. C,D) Single cell RNA-seq and immunohistochemistry of normal human kidneys showing SPTAN1 expression in tubular epithelia.

FIGS. 17A-D. SPTAN1 expression in kidney organoids and human kidneys. (A) Immunohistochemistry for SPTAN1, CDH1, and LTL. SPTAN1 is detected in cytoplasm under cell membranes in CDH1+ tubules in wild-type (WT) kidney organoids cultured under flow. The area marked by a red square is magnified on the right. A scale bar: 20 mm. (B) Immunohistochemistry for SPTAN1 in WT (PKHD1+/+) and ARPKD (PKHD1−/−) organoids cultured under flow. Yellow arrows: CDH1+ tubules. Scale bars: 100 mm (upper side), and 20 mm (lower side). (C) Quantification of SPTAN1 intensity in CDH1+ tubules of WT and ARPKD organoids cultured under flow. Day 22-24 organoids were imaged and quantified. Total 14553 cells (control:5389, mutant:9164) were quantified. **: p<1.0×10⁻⁷. (D) Immunohistochemistry for SPTAN1 in human kidneys of normal and ARPKD patients. A scale bar: 50 mm.

### DETAILED DESCRIPTION

Recent advances in stem cell biology have enabled the generation of mini-organs, known as organoids (1-3). Human organoids recapitulate cell types and 3D tissue architecture found in human organs and have emerged as new platforms for disease modeling in translational medicine (4-6). However, there are still many challenges in replicating the complex physiology of native organs using static organoids, especially for modeling human diseases that require appropriate external stimuli from cellular microenvironments.

ARPKD represents a leading cause of pediatric dialysis dependency and kidney transplantation (10). This disease progresses far more rapidly than human autosomal dominant PKD (ADPKD), conveying significant morbidity and mortality in children. While rodent models have improved the understanding of pathomechanisms in ARPKD (11, 12), it has been challenging to create physiological human models that accelerate translation to patients. ARPKD cysts develop in the distal tubules and collecting ducts in humans (11). By contrast, a renal cystic phenotype is often absent in mouse models with Pkhd1−/−, and when present, is mild and progresses slowly in proximal tubule segments (12). PCK rats generally used to model ARPKD develop cysts in distal nephrons; however, the cyst progression is slow and focal, unlike human ARPKD but rather similar to ADPKD (11, 13). To overcome the limitations of animal models in clinical translation, organ-on-a-chip models are evolving for studying human cell biology under physiological conditions. To date, proximal tubule-on-a-chip models have been developed in planar (14-16) and three-dimensional geometries (15, 16). However, since they are composed of proximal tubule cells seeded onto either a porous membrane or adherent extracellular matrix housed within prefabricated microchannels, they cannot adequately recapitulate tubular enlargement in PKD. As shown herein, a kidney organoids-on-chip platform provided a physiologically relevant model for ARPKD, allowing the identification of mechanosensing signals as key drivers of cystogenesis and new therapeutic options.

The present observations shed light on two important questions regarding the disease mechanisms of ARPKD. The segment-specificity of cyst formation has been an unresolved question for a long time. The segment-specificity of cyst formation differs among species, especially between humans and mice, raising concern about the translatability of animal studies into patient care. Single-cell transcriptomics show that FOS is more localized to distal nephrons in humans compared to mice, where proximal tubules also express FOS. Since mouse ARPKD models develop proximal tubular cysts, FOS might be the crucial determinant of segment-specific cyst formation leading to species-dependent differences.

Our model also provides new insights on why mutations in PKHD1 lead to cyst formation. Transcriptomic analysis demonstrates the converse regulation of genes associated with cytoskeleton remodeling and negative cell cycle regulation when PKHD1-homozygous mutations are introduced to human kidney organoids. However, the upstream molecules, RAC1 and FOS, are not downregulated but indeed upregulated in PKHD1−/− organoids, suggesting PKHD1 is involved in gene expression regulation downstream of RAC1 and FOS.

### Methods of Treatment

Inhibitors of RAC1 and FOS were shown to have therapeutic effects in the present model. Provided herein are methods for treating ARPKD by administering a therapeutically effective amount of an inhibitor of RAC1 or FOS. Generally, the methods include administering a therapeutically effective amount of an inhibitor of RAC1 or FOS as described herein, to a subject who is in need of, or who has been determined to be in need of, such treatment, e.g., a subject who has been diagnosed with ARPKD (e.g., wherein the subject has a mutation in PKHD1 or has been confirmed to have a mutation in PKHD1). ARPKD was previously known as infantile polycystic kidney disease. In some embodiments, the subject does not have cancer.

As used in this context, to “treat” means to ameliorate at least one symptom of ARPKD. Often, ARPKD results in solid tumor cell nests produced by tubular cell hyperplasia and impaired renal function; thus, a treatment comprising administration of a therapeutically effective amount of a compound described herein can result in a reduction in cyst formation or growth, and improved renal function, as evidenced by increased glomerular filtration rate (GFR), reduced serum creatinine level, and/or reduced Blood urea nitrogen (BUN).

Inhibitors of RAC1 include FDA-approved NSAIDs (R-Naproxen and R-Ketorolac; racemic ketorolac can also be used); ZINC69391 and analog 1A-116 (Cardama et al., Onco Targets Ther (2014) 7:2021-33); 1D-142 (Ciarlantini et al., ChemMedChem (2020) 16(6):1011-21); anthraquinone-quinazoline hybrid 7B (Kang et al., Int J Oncol (2021) 58(5):19); NSC23766 (Gao et al., Proc. Natl. Acad. Sci. USA 2004, 101, 7618-7623); thioquinoline derivative EHT 1864 (Shutes et al., J. Biol. Chem. 2007, 282, 35666-35678); GYS32661 (Revere Pharmaceuticals, Boston, MA, USA) and the dual Cdc42/Rac inhibitor MBQ-167 (MBQ Pharma, Puerto Rico, US). See, e.g., Sauzeau et al., Biomedicines 2022, 10(6), 1357.

ARPKD is a developmental disease with a 30% neonatal mortality rate (8, 9). Naproxen is approved for children over 2 years of age permitting efficacy trials in afflicted children (66), though there are concerns to using NSAIDs in patients with kidney disease. Importantly, the present data demonstrates cyst suppression with R-Naproxen, an enantiomer with limited COX inhibition and enhanced RAC1 inhibition (47, 67), lessening concerns regarding NSAID-related kidney side effects.

Inhibitors of FOS include the clinically tested inhibitor T-5224 (3-{5-[4-(cyclopentyloxy)-2-hydroxybenzoyl]-2-[(3-hydroxy-1,2-benzisoxazol-6-yl)methoxy]phenyl}propionic acid). T-5224 is a specific FOS inhibitor proven to be safe in humans (45). The present methods and compositions can use T-5444 or pharmaceutically acceptable salts thereof, as well as analogs thereof such as the benzophenone derivatives described in US20050113400, e.g., comprising the formula

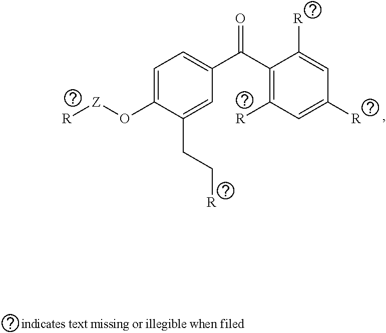

wherein
R1 represents, for example, an optionally substituted heterocyclic group, or a substituted phenyl group; Z represents, for example, an alkylene group;
R2 represents, for example, a carboxyl group optionally protected with alkyl;
R3 represents, for example, an optionally protected hydroxyl group;
R4 represents, for example, an optionally substituted cycloalkyloxy group; and
R5 represents, for example, a hydrogen atom,
   or a pharmaceutically acceptable salt thereof.

### Inhibitory Oligonucleotides Targeting RAC1 or FOS

As described above, the methods can include the administration of inhibitory oligonucleotides (“oligos”) targeting RAC1 or FOS (i.e., RAC1 or FOS mRNA or DNA) that reduce RAC1 or FOS expression. Oligos useful in the present methods and compositions include antisense oligonucleotides, ribozymes, external guide sequence (EGS) oligonucleotides, siRNA compounds, single- or double-stranded RNA interference (RNAi) compounds such as siRNA compounds, molecules comprising modified bases, locked nucleic acid molecules (LNA molecules), antagomirs, peptide nucleic acid molecules (PNA molecules), mixmers, gapmers, and other oligomeric compounds or oligonucleotide mimetics that hybridize to at least a portion of RAC1 or FOS and modulate its function. In some embodiments, the oligos include antisense RNA, antisense DNA, chimeric antisense oligonucleotides, antisense oligonucleotides comprising modified linkages, interference RNA (RNAi), short interfering RNA (siRNA); or a short, hairpin RNA (shRNA); mixmers; gapmers; or combinations thereof. See also WO 2015/051239.

Sequences for human RAC1 or FOS are known in the art and include the following.

| Target | Nucleic Acid | Protein | Name/Isoform |
|---|---|---|---|
| FOS | NM_005252.4 | NP_005243.1 | protein c-Fos |
| RAC1 | NM_006908.5 | NP_008839.2 | ras-related C3 botulinum toxin |
| | | | substrate 1 isoform Rac1 |
| | NM_018890.4 | NP_061485.1 | ras-related C3 botulinum toxin |
| | | | substrate 1 isoform Rac1b* |

*Variant Rac1b includes an alternatively spliced 57 bp region (exon 3b) that is missing in transcript variant Rac1.

Exemplary genomic sequence for human RAC1 is at NG_029431.1, range 5033 to 34473, and for FOS is at NG_029673.1, range 5051 to 8453.

In some embodiments, the oligos hybridize to at least 7, 8, 9, 10, 11, 12, 13, 14, 15, 16, 17, 18, 19, 20, 21, 22, 23, 24, 25, 26, 27, 28, 29, 30 or more consecutive nucleotides of the target sequence.

In some embodiments, the methods include introducing into the cell an oligo that specifically binds, or is complementary, to RAC1 or FOS. A nucleic acid that binds “specifically” binds primarily to the target, i.e., to RAC1 or FOS RNA but not to other non-target RNAs. The specificity of the nucleic acid interaction thus refers to its function (e.g., inhibiting RAC1 or FOS) rather than its hybridization capacity. Oligos may exhibit nonspecific binding to other sites in the genome or other mRNAs, without interfering with binding of other regulatory proteins and without causing degradation of the non-specifically-bound RNA. Thus this nonspecific binding does not significantly affect function of other non-target RNAs and results in no significant adverse effects. These methods can be used to treat a subject, e.g., a subject at risk for neurodegeneration following acute injury or with evidence of a chronic neurodegenerative disease, by administering to the subject a composition (e.g., as described herein) comprising an oligo that binds to RAC1 or FOS. Examples of RAC1 or FOS target sequences are provided above.

In some embodiments, the methods described herein include administering a composition, e.g., a sterile composition, comprising an oligo that is complementary to RAC1 or FOS sequence as described herein. Oligos for use in practicing the methods described herein can be an antisense or small interfering RNA, including but not limited to an shRNA or siRNA. In some embodiments, the oligo is a modified nucleic acid polymer (e.g., a locked nucleic acid (LNA) molecule), a gapmer, or a mixmer.

Oligos have been employed as therapeutic moieties in the treatment of disease states in animals, including humans. Oligos can be useful therapeutic modalities that can be configured to be useful in treatment regimens for the treatment of cells, tissues and animals, especially humans.

For therapeutics, an animal, preferably a human, suspected of having or being at risk of neurodegeneration is treated by administering an oligo in accordance with this disclosure. For example, in one non-limiting embodiment, the methods comprise the step of administering to the animal in need of treatment a therapeutically effective amount of an oligo as described herein.

In some embodiments, the oligos are 10 to 50, 13 to 50, or 13 to 30 nucleotides in length. One having ordinary skill in the art will appreciate that this embodies oligonucleotides having antisense (complementary) portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. It is understood that non-complementary bases may be included in such oligos; for example, an oligo 30 nucleotides in length may have a portion of 15 bases that is complementary to the targeted RAC1 or FOS RNA. In some embodiments, the oligonucleotides are 15 nucleotides in length. In some embodiments, the antisense or oligonucleotide compounds of the invention are 12 or 13 to 30 nucleotides in length. One having ordinary skill in the art will appreciate that this embodies oligos having antisense (complementary) portions of 12, 13, 14, 15, 16, 17, 18, 19, 20, 21, 22, 23, 24, 25, 26, 27, 28, 29 or 30 nucleotides in length, or any range therewithin.

Preferably the oligo comprises one or more modifications comprising: a modified sugar moiety, and/or a modified internucleoside linkage, and/or a modified nucleotide and/or combinations thereof. It is not necessary for all positions in a given oligonucleotide to be uniformly modified, and in fact more than one of the modifications described herein may be incorporated in a single oligonucleotide or even at within a single nucleoside within an oligonucleotide.

In some embodiments, the oligos are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric oligos of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5, 220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the oligo comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NH—O—CH₂, CH, ~N(CH₃)—O—CH₂ (known as a methylene(methylimino) or MMI backbone], CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH,); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

### Pharmaceutical Compositions and Methods of Administration

The methods described herein include the use of pharmaceutical compositions comprising or consisting of inhibitors of RAC1 and/or FOS as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions. In some embodiments, no other active compounds are present.

Pharmaceutical compositions are typically formulated to be compatible with its intended route of administration. Examples of routes of administration include parenteral, e.g., intravenous, intradermal, subcutaneous, oral or mucosal (e.g., sublingual or inhalation), transdermal (topical), and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### Dosage

An “effective amount” is an amount sufficient to effect beneficial or desired results. For example, a therapeutic amount is one that achieves the desired therapeutic effect. This amount can be the same or different from a prophylactically effective amount, which is an amount necessary to prevent (reduce risk of or delay) onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Clinical studies can be performed to discover optimal and safe dosages of drugs including R-Ketorolac, R-Naproxen and T-5224 that slow cyst progression without significant risk of renal toxicity in ARPKD patients of all ages. An exemplary dose of racemic ketorolac of 15 or 30 mg achieved likely therapeutic ranges of R-ketorolac, as reported in Guo et al., Clin Cancer Res. 2015 Nov. 15; 21(22): 5064-5072.

### EXAMPLES

The invention is further described in the following examples, which do not limit the scope of the invention described in the claims.

### Materials and Methods

The following materials and methods were used in the Examples below

### Cell Culture and Maintenance

H9 (WiCell) human ES cells, H9-derived PKHD1 mutants, and ARPKD patient-derived iPSCs (68) were maintained on hESC-qualified Geltrex (Thermo Fisher Scientific) coated plates using StemFit® Basic02 (Ajinomoto Co., Inc.) supplemented with 10 ng/ml of FGF2 (Peprotech), as previously reported (69).

### Nephron Organoid Differentiation

Nephron organoids were differentiated and maintained as established in previous protocols (1, 69). Quality check was performed by immunostaining for SIX2 on day 8 and for LTL, CDH1, and PODXL, segment-specific markers of nephron on day 21. For static culture, the basal medium consisting of Advanced RPMI (ARPMI, Thermo Fisher) and GlutaMAX (Thermo Fisher) was changed every 2-3 days.

Generation of Heterozygous and Homozygous PKHD1 Mutant hPSCs

Heterozygotic (PKHD1+/−) and compound homozygotic (PKHD1−/−) mutant lines were generated using methodology previously outlined (70), which generated clones of isogenic progeny via clonal expansion from single cells. PX459 plasmid encoding a Streptococcus pyogenes Cas9 and guide RNA (sgRNA) targeting the third exon of PKHD1 (5′-TCATTTTTGATGGTAGGTTG-3′; SEQ ID NO:1) was transfected into H9 hESCs to generate the heterozygotic mutant line, which then served as the parental line to generate the compound homozygotic mutant. Transfected cells were selected by transient puromycin treatment. CRISPR/Cas9-induced mutations of individual clones were identified by deep sequencing.

### Targeted Deep Sequencing

Each target area was PCR amplified from the isolated single clones for next generation sequencing (NGS) targeted deep sequencing at the Massachusetts General Hospital Center for Computational & Integrative Biology DNA Core (MGH CCIB DNA Core, Cambridge MA). Briefly, target-specific primers were designed to amplify 200-280 bp from each iPSC clone, including the target site of CRISPR modification, and subjected to NGS deep sequencing. CRISPR-induced variants were determined by alignment to the public reference genome for comparison to unmodified iPSC clones.

### Engineered Extracellular Matrix Preparation

The extracellular matrix (ECM) was prepared as previously described (15, 19, 71). The matrix contained 300 bloom type A gelatin from porcine skin (Sigma Aldrich) and fibrinogen (Millipore Sigma), which were enzymatically crosslinked using transglutaminase (Moo Glue, The Modernist Pantry) and thrombin (MPI Biologicals), respectively. Specifically, a solution of 2 wt % gelatin, 10 mg/ml fibrinogen, 2.5 mM calcium chloride, and 2 mg/ml transglutaminase in PBS was first prepared. The solution was mixed well and incubated at 37 °C for 15 min. Following incubation, 1 ml of the solution was quickly mixed with thrombin by fast pipetting resulting in a final thrombin concentration of 2 U/ml. The solution was then quickly cast onto the millifluidic chip (see below) and distributed evenly over the surface. This ECM solution gelled in ~5 min at RT, and was further cured for 3 hr at 37 °C in a cell culture incubator in a sterile container.

### Millifluidic Chip Fabrication

Perfusion chip devices consisted of a 10 ml syringe medium reservoir (Nordson EFD), a leur lock connection nozzle (Nordson EFD), inlet perfusion tubing (2-stop Pharmed BPT tubing, Ismatech, Cole Parmer), perfusion pins (18 G, Nordson EFD), a custom machined stainless-steel base, a 5 mm acrylic lid, outlet perfusion tubing (Cole-Parmer Peroxide-Cured Silicone Tubing, 1/32″ID×3/32″OD), 4×12 mm M4 screws (McMaster Carr) and a custom-designed millifluidic chip. The millifluidic chips were fabricated as previously described (19). Briefly, the millifluidic chip gasket was printed onto a 50×75×1 mm glass slide by a multi-material 3D printer (AGB 10000, Aerotech Inc) with a polydimethylsiloxane (PDMS) ink with a base-to-crosslinker ratio of 10:1 (SE1700, Dow Corning). A custom-written MatLab code was used to produce the G-code needed to print each gasket onto the top surface of a glass slide. After printing, each PDMS gasket was cured at 80 °C for 2 hr. Next, the gasket was filled with a 1 mm thick layer of ECM, as described above. To assemble the perfusion device, the medium reservoir was connected to the inlet and outlet tubing with a sterile syringe filter in line and the millifluidic chip via the perfusion pins. The chip was placed onto the stainless-steel base, and the culture medium was allowed to advance via hose-pinch clamps in the gasket until ~¾ of the hydrogel ECM was covered. Subsequently, the kidney organoids were placed into the chip and allowed to settle onto the ECM. Following the seeding of the kidney organoids, the lid was carefully placed on top of the gasket and secured in place by 4 screws. The chip was filled with culture medium and then placed inside of a cell culture incubator.

For live imaging of kidney organoids under flow conditions, the PDMS gasket was printed onto a 50 mm×75 mm×3 mm thick acrylic substrate with a laser-cut rectangular window (32 mm×45 mm) that was covered using a #1 cover glass (35 mm×50 mm) and secured in place with cyanoacrylate. The PDMS was cured for 3 days at RT to avoid warping of the acrylic base. The millifluidic chip was assembled and filled with ECM. Kidney organoids were then placed on top of the ECM and cultured, as described above. For live imaging, the modified kidney organoids-on-chip assembly was placed onto the stage of a confocal microscope, with the objective was located under the imaging window.

### Culturing Kidney Organoids-On-a-Chip

The organoids were seeded onto the chip devices on day 14 of their differentiation and cultured on chip by flowing cell culture medium composed of ARMPI with GlutaMAX, 1% Antibiotic/Antimycotic solution (Gibco), and 1.5% heat-inactivated fetal bovine serum (Gibco). All cell culture medium was pre-equilibrated to the cell culture incubator environment for at least 2 hr before chip assembly and seeding. The kidney organoids-on-a-chip was then held in a cell culture incubator for 30 min under static conditions, before connecting the inlet perfusion tubing to an IPC-N peristaltic pump (Ismatech) and perfusing the cell culture media at 42.7 μl/min overnight in the incubator. The following day (day 15 of organoid differentiation), the sterile filter was removed from the perfusion line to avoid bubble formation at high perfusion speeds, and the perfusion speed was ramped up to 4.27 mL/min. Cell culture medium was changed in the reservoir every 2-3 days.

### Forskolin Treatment of Kidney Organoids in Static Culture

Kidney organoids were cultured in static condition and treated with 20 μM of forskolin (Sigma, #F6886), a cAMP inducer, from day 14 to day 35.

### Cyclic AMP ELISA Experiment

Kidney organoids were harvested on day 35 of differentiation and cAMP measurement was determined using Cyclic AMP Competitive ELISA kit (Sigma, EMSCAMPL) according to the manufacturer's instructions. The optical density was read at 405 nm by Synergy H1 plate reader (BioSPX) and assessed by Gen5 software (BioTeK).

siRNA Transfection

siKIF3A (Thermo Fisher, Silencer Select ID s21942) and siControl (Thermo Fisher, Silencer Select Negative Control No. 1) were obtained. According to the manufacturer's instructions, the initial siRNA transfection was performed with Lipofectamine RNAiMax (Thermo Fisher) on day 8 of differentiation (corresponding to the nephron progenitor cell stage). Organoids on day 16 of differentiation were transfected again with siKIF3A and siControl in culture media containing 1.5% heat-inactivated fetal bovine serum without antibiotics on chip (72). The medium was replaced 48 h after each transfection.

### FliptR and Live Nuclear Dye Labeling

Kidney organoids were seeded on modified chips (for living imaging) on day 14 of their differentiation and cultured under flow. FliptR (Spirochrome) and SPY700-DNA (Spirochrome) staining solution were applied to the organoids on day 18-21 of differentiation according to manufacturer's instructions and incubated at 37 °C in a humidified atmosphere containing 5% CO2 for 15 min before imaging.

### Lifetime Measurements

FLIM imaging was performed using Leica STELLARIS 8 confocal microscopy. Excitation was performed using a pulsed 488 nm pulsed laser, and the emission signal was collected through a 575-650 nm bandpass filter. LAS X FLIM/FCS software (Leica) was then used to fit fluorescence decay data (from full images or regions of interest in organoid tubules). Fitted lifetime data were expressed as means±s.d. Δτ₂ was calculated with the equation Δτ₂=τ₂-average τ₂ during perfusion or rest.

### Drug Screening Tests of Kidney Organoids

To determine optimal concentration of each compound, PKHD1-mutant organoids were treated with the following concentration from day 16 to day 35 of differentiation: T-5224 (Thermo Fisher Scientific, 50-115-1835) at 5, 10, and 20 μM, Z-DEVD-FMK at 1, 10, and 50 μM (R&D systems, FMK004), Quercetagetin at 10, 40, and 200 μM (Millipore Sigma, 551590), NSC23766 at 25, 50, and 100 μM (Abcam, ab142161), rhCXCL16 at 5, 10, and 25 ng/ml (R&D systems, 976-CX-025), 2-MeOE₂ at 1, 5, and 10 μM (Selleckchem, S1233), R-Naproxen at 10, 20, 200, and 400 μM (Millipore Sigma, 82170), and R-Ketorolac at 0.5, 1, 10, and 20 μM (Millipore Sigma, 1654) (45, 46, 73-77). Full media exchanges were conducted every 2-3 days. Treatment of the kidney organoids-on-a-chip was started at day 16 of their differentiation until day 35. The compounds were added to the medium reservoir at every medium change in the following concentrations: T-5224 at 10 μM, Z-DEVD-FMK at 1 μM, Quercetagetin at 10 μM, NSC23766 at 10 μM, rhCXCL16 at 1 ng/ml, 2-MeOE₂ at 5 μM, R-Naproxen at 20 μM, S-Naproxen at 20 μM, and R-Ketorolac at 1 and 10 μM.

### Immunocytochemistry of 2D Tissues

Nephron progenitor cells on day 8 of differentiation were rinsed with PBS and fixed with 4% paraformaldehyde (PFA) for 20 min at RT. Fixed cells were washed three times in PBS and blocked with blocking buffer (BB; 5 wt % donkey serum in PBS containing 0.3 wt % TritonX-100) for 1 hr at RT. The cells were then incubated with anti-SIX2 (Proteintech, 11562-1-AP) in antibody dilution buffer (ADB; 1% BSA containing 0.3% TritonX-100) overnight at 4 °C. After washing three times in PBS, the cells were incubated with Alexa Fluor secondary antibodies (Life Technologies) in ADB for 1 hr at RT. DAPI (Sigma, #D8417) was added to stain the nucleus. Immunofluorescence was photographed using a Nikon C1 confocal microscopy or a Leica STELLARIS 8 confocal microscopy.

### Immunocytochemistry of Kidney Organoids

Nephron organoids were fixed with 4% PFA for 1 hr at RT, incubated in 30% sucrose overnight at 4 °C, embedded in O.C.T., and 10 μm frozen sections were cut using a cryostat. Samples were blocked with BB for 1 hr at RT. and then incubated with primary antibodies in ADB overnight at 4 °C. The following antibodies were used in these studies: anti-CDH1 (Abcam, ab11512), anti-LTL (Vector lab, B-1325), anti-PODXL (R&D systems, AF1658), anti-TUBA1A (Abcam, ab179484), anti-K167 (Dako, m7248), anti-ATP1A1 (Abcam, ab76020), anti-PKHD1 (Abcam, ab204951(8G12A1)), anti-ARFIP2 (Bioss, BS-1442R), anti-PRDM1 (Novus Biologicals, NB600-235SS), anti-GATA3 (R&D systems, AF2605), anti-BRN1 (Santa Cruz, sc-6028-R), anti-KIM-1 (R&D systems, AF1750), and anti-phospho-histone H2AX (Cell Signaling, #2577). For immunostaining with biotinylated LTL, Streptavidin/Biotin Blocking Kit (Vector Labs, #SP-2002) was used according to the manufacturer's protocol. After washing three times in PBS, samples were incubated with Alexa Fluor secondary antibodies in ADB for 1 hr at RT. Vectashield with DAPI (Vector labs) was added to stain the nucleus. For Phalloidin staining, Acti-Stain 555 Phalloidin (Cytoskeleton, Inc., #PHDH1-A) was used according to the manufacturer's protocol. Images were taken using a Nikon C1 confocal microscopy or a Leica STELLARIS 8 confocal microscopy.

### Whole-Mount Immunohistochemistry

Nephron organoids on day 21 of differentiation were fixed with 4% PFA for 1 hr at RT. Samples were blocked with BB for 1 hr at RT and then incubated with primary antibodies in ADB overnight at 4 °C. The following antibodies were used in these studies: anti-CDH1 (Abcam, ab11512), anti-LTL (Vector lab, B-1325), and anti-PODXL (R&D systems, AF1658). For immunostaining with biotinylated LTL, Streptavidin/Biotin Blocking Kit (Vector Labs, #SP-2002) was used according to the manufacturer's protocol. After washing three times in PBS, samples were incubated with Alexa Fluor secondary antibodies in ADB for 1 hr at RT. Vectashield with DAPI (Vector labs) was added to stain the nucleus. Images were taken using a Nikon C1 confocal microscopy or a Leica STELLARIS 8 confocal microscopy.

Whole-Mount Immunohistochemistry with Sample Clearing

The chip device was taken out of the incubator for fixation, and the lid was carefully removed in a sterile biosafety cabinet. The kidney organoids adherent on the surface of the ECM were rinsed with PBS, carefully overlayed with 4% PFA, and fixed for 45 min at RT. Post fixation, the samples were washed three times with 0.1% Triton-X (Sigma) for 30 min. Organoids were stained and cleared with ethyl cinnamate (Sigma), as described before (78). The following antibodies were used in these studies: anti-CDH1 (Abcam, ab11512), anti-CFTR (Sigma, HPA021939), anti-LTL (Vector lab, B-1325), anti-PODXL (R&D systems, AF1658), anti-K167 (Dako, m7248), Anti-pS6RP (Cell signaling, 2211S), anti-RAC1 (Developmental Studies Hybridoma Bank, CPTC-RAC1-2), anti-FOS (Abcam, ab190289). Samples were examined by a Zeiss TIRF/LSM 710 confocal microscopy or a Leica STELLARIS 8 confocal microscopy, and by using Imaris 3D software (Bitplane).

### Immunostaining and Lectin Staining of Human Tissue

Immunohistochemistry of human ARPKD samples and normal portions of tumor nephrectomy was carried out as described previously (75, 79). Briefly, 4 μm-thickness paraffin sections were blocked with 3% H₂O₂ and 1% BSA-PBS, after deparaffinization, hydration, and antigen retrieval in 0.01 M citrate buffer, pH6.0 at 95 °C for 30 min. The sections were incubated overnight at 4 °C with primary antibodies: anti-human Rac1-GTP Ab (26903, 1:100, NewEast Biosciences) or anti-cFOS Ab (Ab190289, 1:2000, Abcam). After washing with PBS 3 times, secondary reagents (Envision+ Rabbit IgG, DAKO, or HRP-Goat anti-Mouse IgM Antibody, Invitrogen) were applied and incubated at room temperature for 1 hour and visualized with ImmPACT DAB (Vector Laboratories). The sections only for Rac1-GTP were counterstained with hematoxylin. The staining was evaluated in at least 500 cystic epithelial cells. The staining of biotin-labeled Lotus Tetragonolobus Lectin (Biotin-LTL; B-1325, 1:200, Vector Laboratories) was performed by a similar method, but without secondary reagent and with an additional blocking for endogenous streptavidin/biotin. This study was performed under the protocol (STUDY00001229) approved by Cedars-Sinai Medical Center Institutional Review Board.

### Dextran Perfusion Assay

Organoids cultured on a chip were treated with 5 g/ml of WGA conjugated-647 (Thermo Fisher, W32466), incubated at 37 °C for 30 min under flow, and rinsed with live cell imaging solution (Thermo Fisher, A14291DJ) once. 10 g/ml of LMD (Thermo Fisher, D3306) was then perfused by gravity. Samples were examined by a Leica STELLARIS 8 confocal microscopy.

### DNA Microarray and Functional Analysis

Total RNA was isolated from kidney organoids with TRIzol (Thermo Fisher) from 3 biological replicates in each condition (PKHD1−/− Flow, forskolin treatment, or Static, and PKHD1+/− Flow, forskolin treatment, or Static). The quality of purified total RNA was analyzed with Agilent 2100 Bioanalyzer (Agilent Technologies). Total RNA was amplified and labeled using an Amino Allyl MessageAmp™ II aRNA Amplification kit (Thermo Fisher). Each sample of aRNA labeled with Cy5 was hybridized with a highly sensitive DNA chip, 3D-Gene® Human Oligo chip 25 k (Toray Industries). Hybridization signals were scanned and analyzed using 3D-Gene® Scanner (Toray Industries). All data were scaled by global normalization. The function of flow-associated DEGs in PKHD1+/− organoids was annotated and analyzed according to the three organizing principles of Gene Ontology (Biological Process, Molecular Function, Cellular Component) by MetaCore™ analysis software (Clarivate). An integrated pathway enrichment analysis was performed using MetaCore™ analysis software. The heat map was made by ExAtlas software kolab.elixirgensci.com/exatlas/).

### Quantitative RT-PCR

Total RNA was isolated from kidney organoid samples using TRIzol (Invitrogen, #15596026). cDNA was synthesized from 500 ng of RNA using a High-capacity cDNA Reverse Transcription kit (Applied Biosystems, #4368814) according to the manufacturer's instructions. Real-time PCR was performed using iTaq SYBR green supermix (Bio-Rad, #1725124) and QuantStudio3 Real-time PCR systems. The expression of mRNA was assessed by evaluating threshold cycle (CT) values. The CT values and relative expression levels were normalized by ribosomal protein S18 (RPS18), and the relative amount of mRNA specific to each of the target genes was calculated (n=3 in each experiment). The primer sequences used for qRT-PCR are listed in Table 1.

TABLE 1
Primer Sequences

| Gene | Forward | # | Reverse | # |
|---|---|---|---|---|
| RPS18 | GCGGCGGAAAATAGCCTTTG | 2 | GATCACACGTTCCACCTCATC | 3 |
| DUSP1 | GGCCATTGACTTCATAGACTCCA | 4 | AACTCAAAGGCCTCGTCCAG | 5 |
| NPTX2 | ACGGGCAAGGACACTATGG | 6 | ATTGGACACGTTTGCTCTGAG | 7 |
| PRDM1 | AAGCAACTGGATGCGCTATGT | 8 | GGGATGGGCTTAATGGTGTAGAA | 9 |
| ARFIP2 | ATCAGAACGATTTGGTCGAGGC | 10 | ATACTTGCGCTTCGTCTCACG | 11 |
| BAIAP2 | GACGTATGCAGCCAAAGGCTA | 12 | GCAGCTCGTTGTGAAAAGACT | 13 |

#, SEQ ID NO:

### Single Cell RNA-Seq

Uniform manifold approximation and projection (UMAP) was made by using KPMP: DK114886, DK114861, DK114866, DK114870, DK114908, DK114915, DK114926, DK114907, DK114920, DK114923, DK114933, and DK114937. Abbreviations in UMAP are as follows: PC, Principal cell; tPC-IC; Principal-intercalated cell (transitional); CNT-IC-A, Connecting tubule intercalated cell type A; IC-A, Intercalated cell type A; VSMC/P, Vascular smooth muscle cell/Pericyte; MON, Monocyte; MDC, Monocyte-derived cell; CNT-PC, Connecting tubule principal cell; aTAL2, Thick ascending limb cell cluster 2 (adaptive/maladaptive/repairing); dCNT-PC, Connecting tubule principal cell (degenerative); MAC-M2, M2-macrophage; cDC, Classical dendritic cell; dPC, Principal cell (degenerative); PEC, Parietal epithelial cell; ncMON, Non-classical monocyte; dC-TAL, Cortical thick ascending limb cell (degenerative); FIB, Fibroblast; dVSMC, Vascular smooth muscle cell (degenerative); DTL1, Descending thin limb cell type 1; dEC-PTC, Peritubular capillary endothelial cell (degenerative); pDC, Plasmacytoid dendritic cell; EC-AEA, Afferent/Efferent arteriole endothelial cell; B, B cell; dCNT, Connecting tubule cell (degenerative); C-TAL; Cortical thick ascending limb cell; T-CYT, Cytotoxic T cell; dDCT, Distal convoluted tubule cell (degenerative); DCT1, Distal convoluted tubule cell type 1; cycEC, Endothelial cell (cycling); cycEPI, epithelial cell (cycling); aFIB, Fibroblast (adaptive/maladaptive/repairing); EC-GC, Glomerular capillary endothelial cell; dIC-A, Intercalated cell type A (degenerative); IC-B, Intercalated cell type B; EC-LYM, Lymphatic endothelial cell; M-TAL, Medullary thick ascending limb cell; MC, Mesangial cell; cycMNP, Mononuclear phagocyte (cycling); MyoF, Myofibroblast; NK1, Natural killer cell type 1; NK2, Natural killer cell type 2; NKT, Natural killer T cell; EC-PTC, Peritubular capillary endothelial cell; PL, Plasma cell; POD, Podocyte; aPT, Proximal tubule epithelial cell (adaptive/maladaptive/repairing); dPT, Proximal tubule epithelial cell (degenerative); dPT/DTL, Proximal tubule epithelial cell/Descending thin limb cell (degenerative); PT-S1/S2, Proximal tubule epithelial cell segment 1/segment 2; PT-S3, Proximal tubule epithelial cell segment 3; T-REG, Regulatory T cell; REN, Renin-positive juxtaglomerular granular cell; T, T cell; cycT, T cell (cycling); aTAL1, Thick ascending limb cell cluster 1 (adaptive/maladaptive/repairing).

### Statistical Analysis and Reproducibility

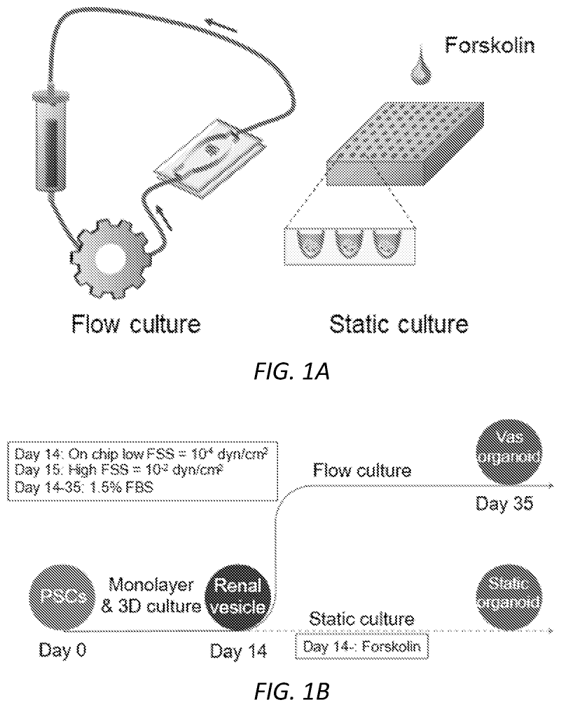

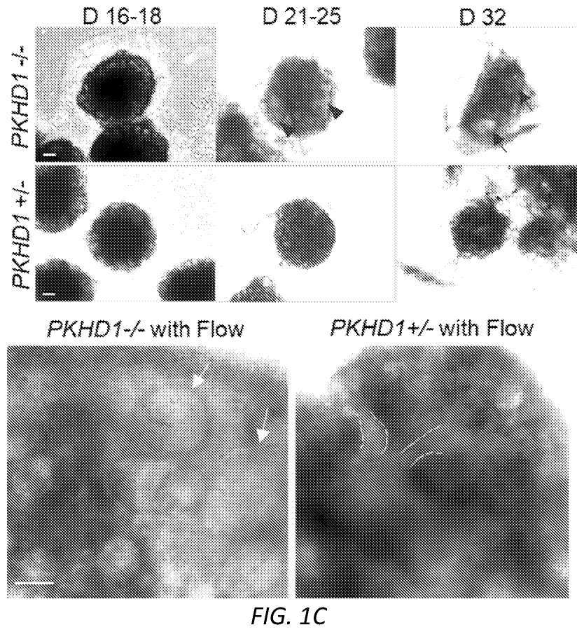

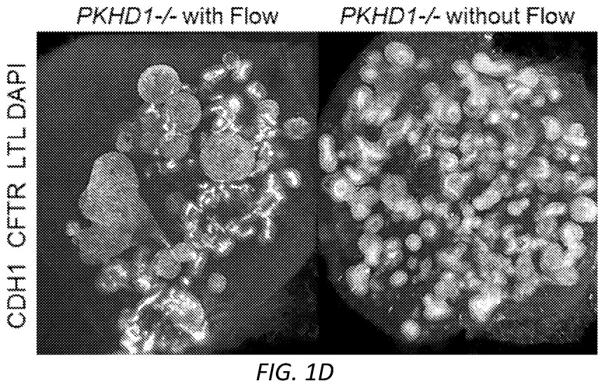

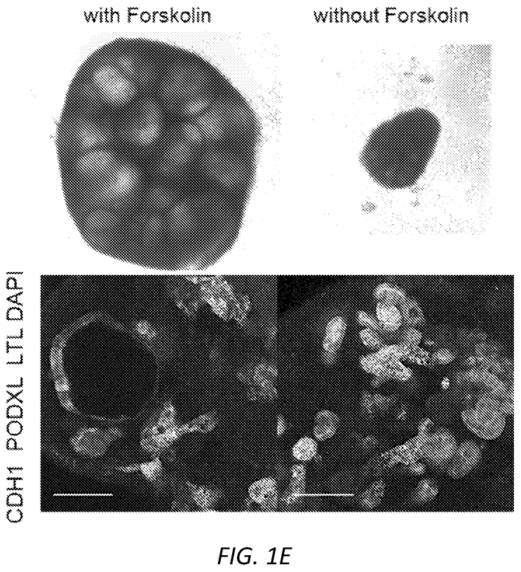

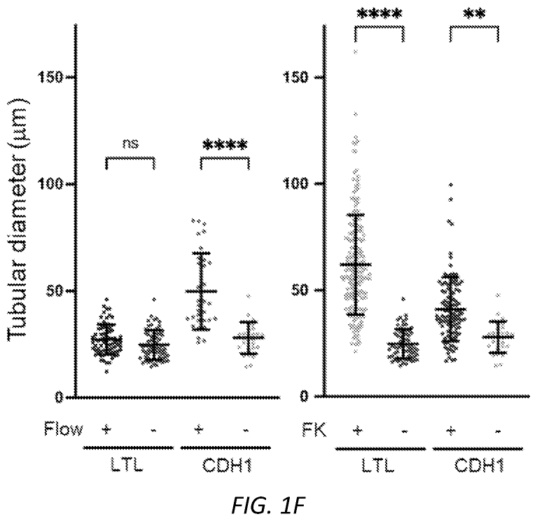

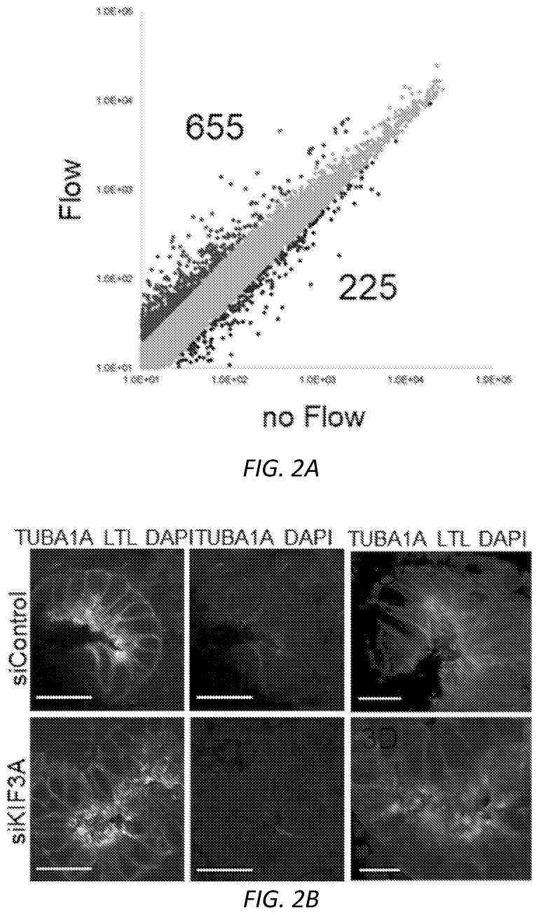

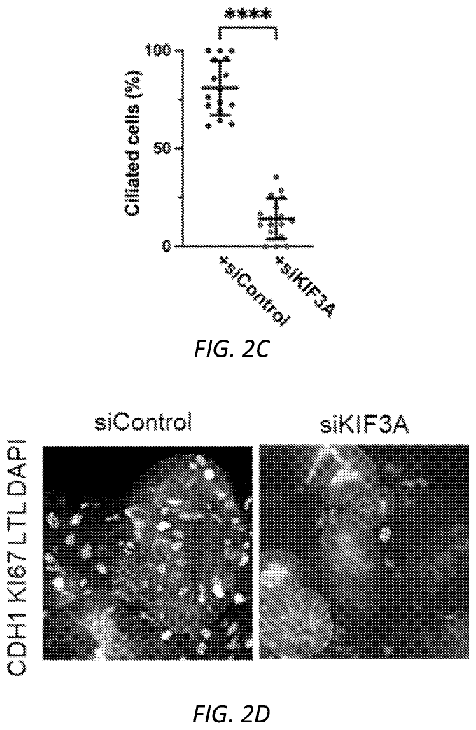

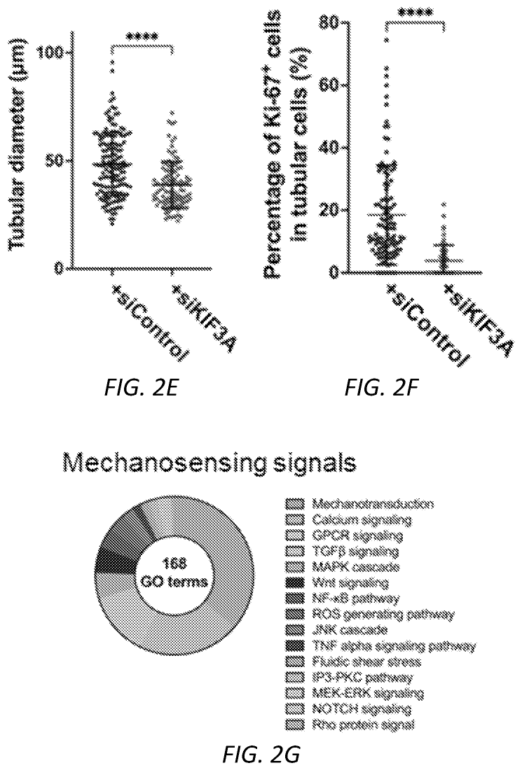

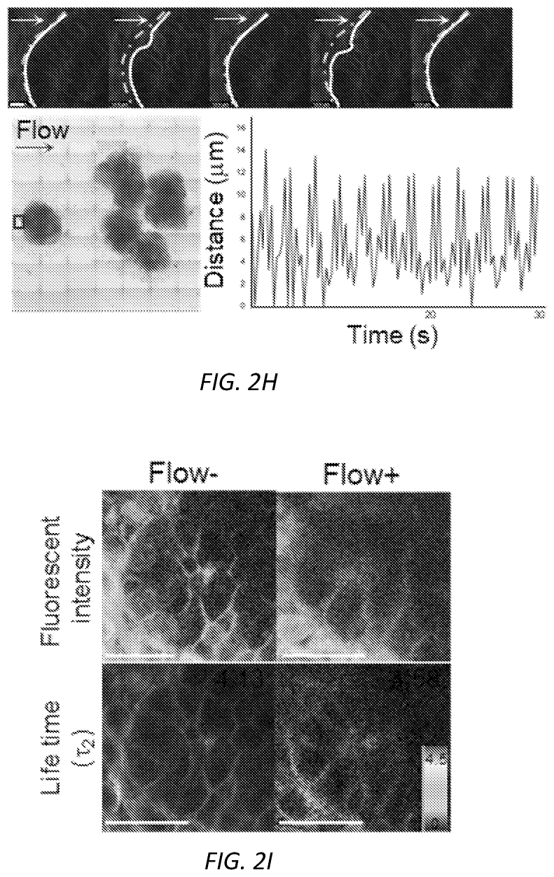

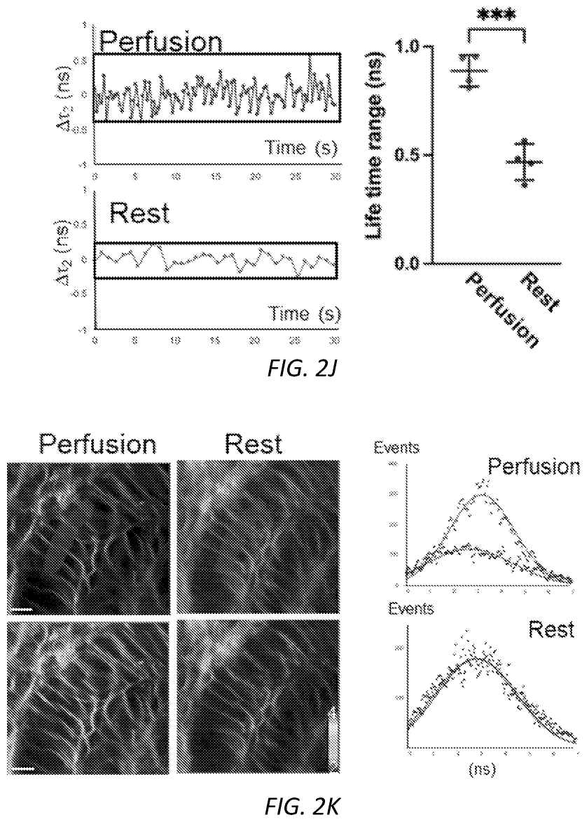

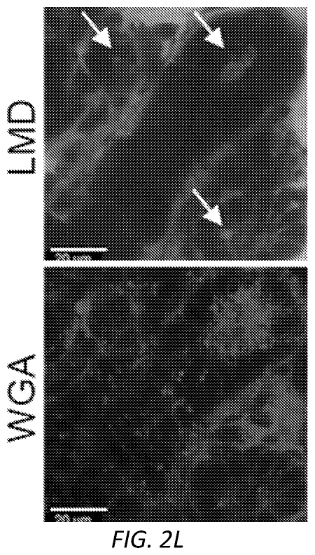

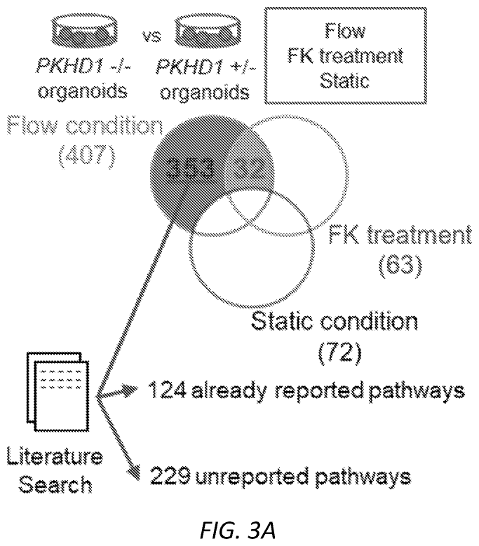

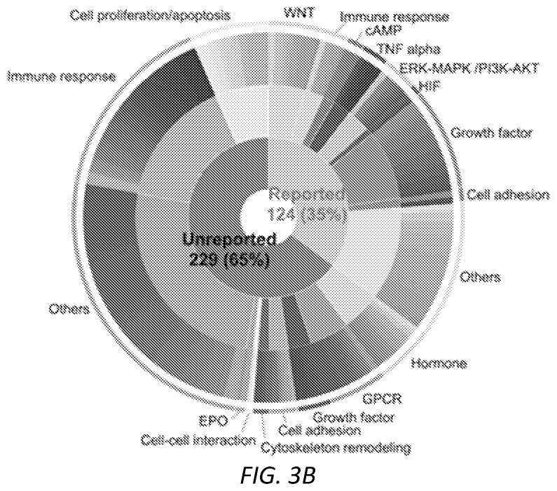

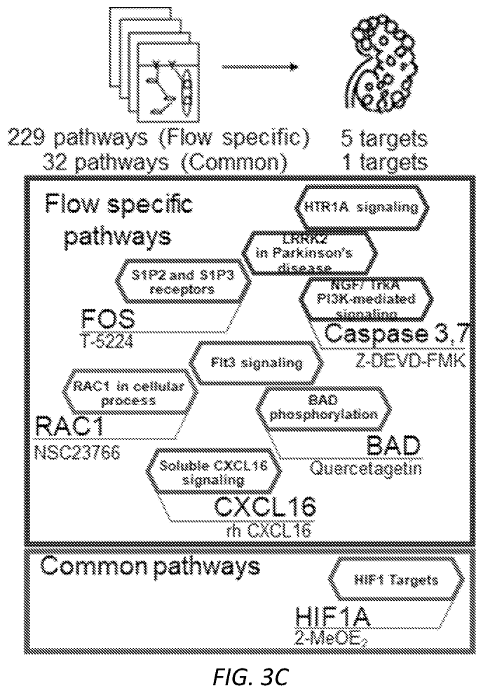

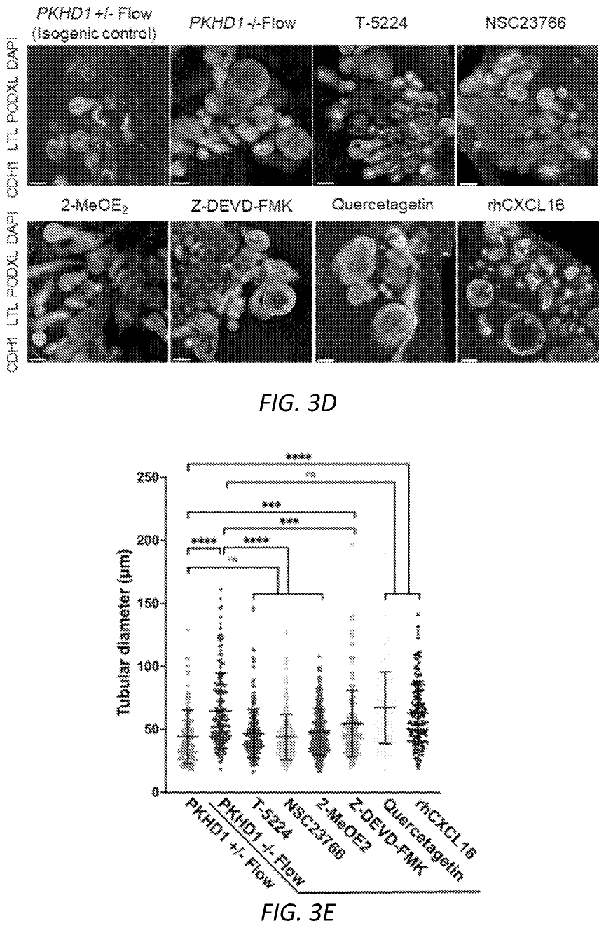

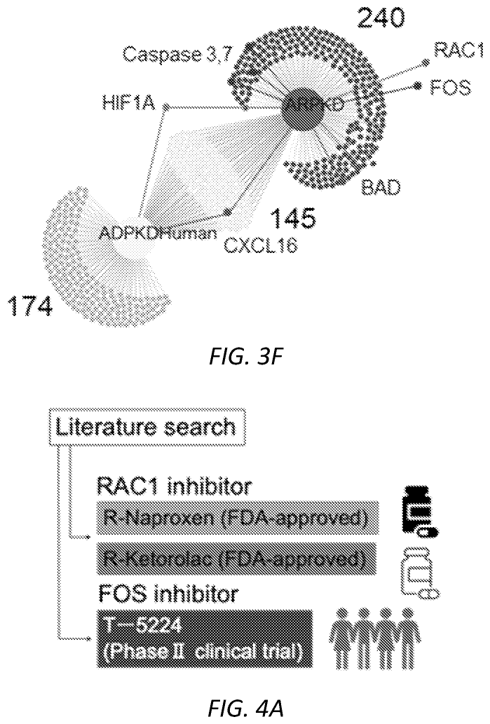

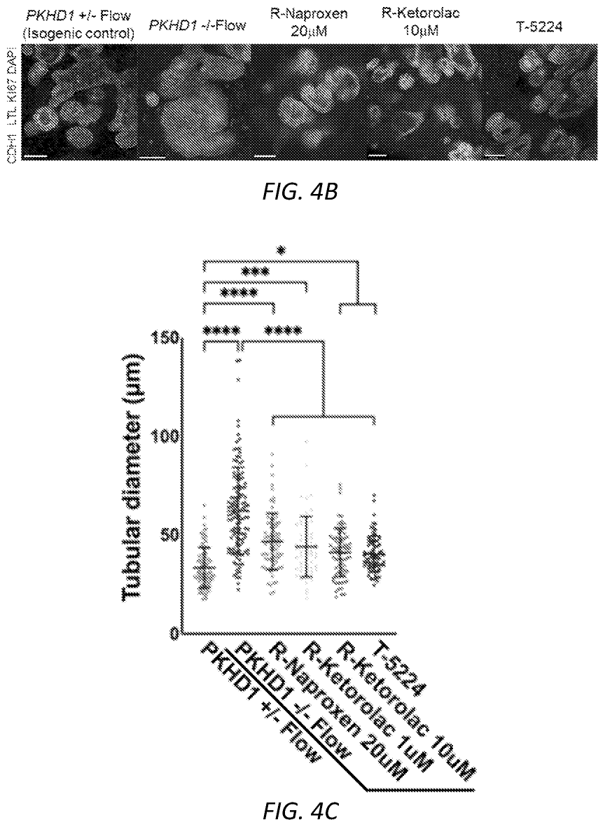

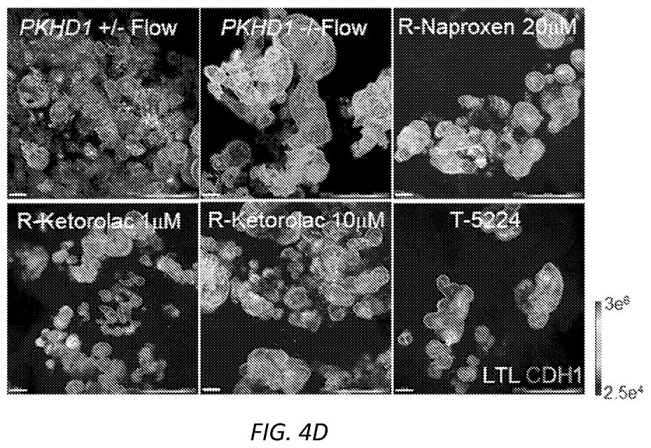

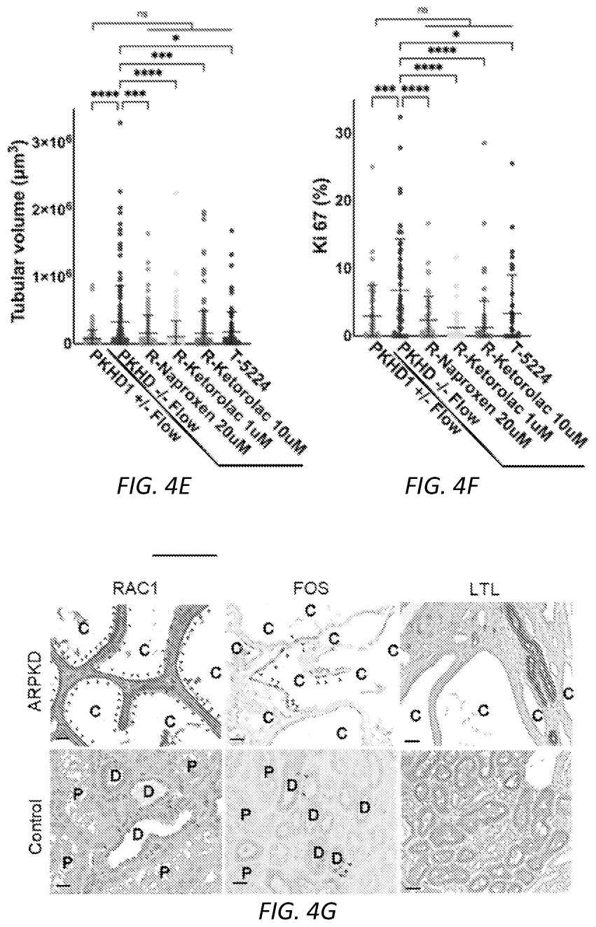

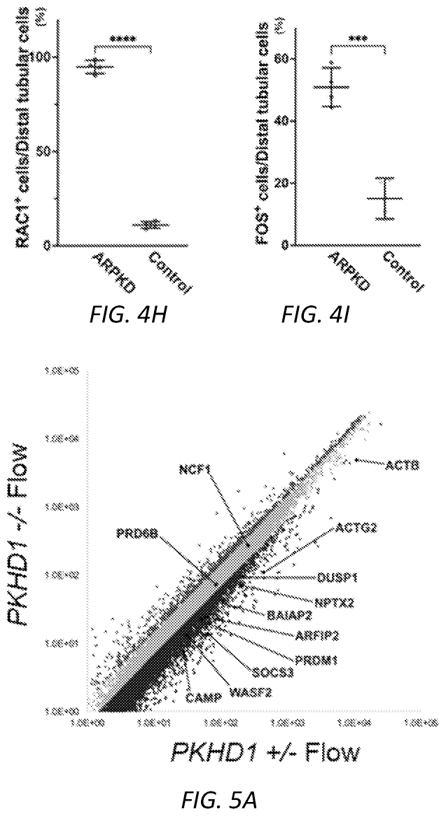

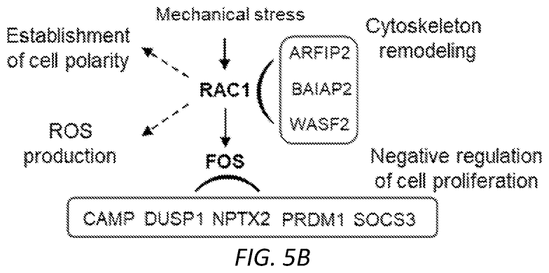

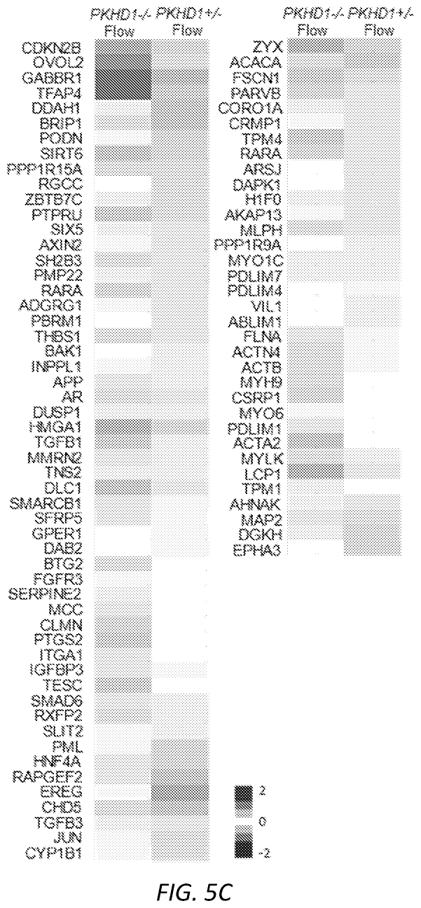

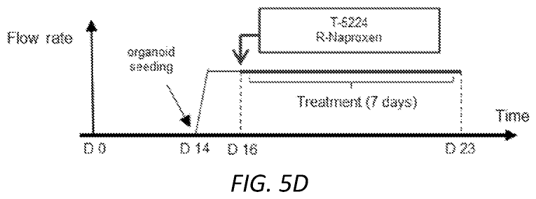

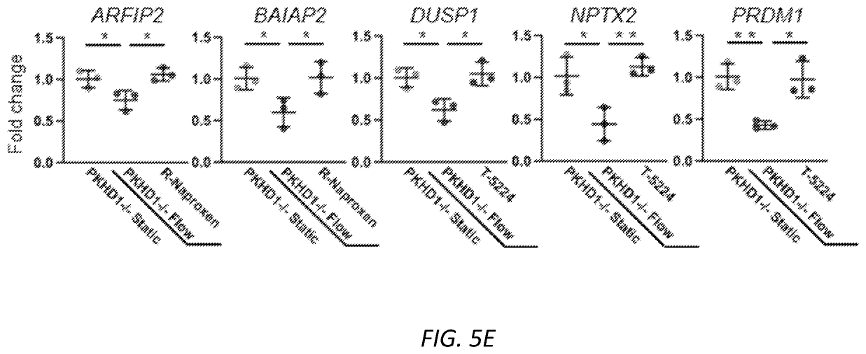

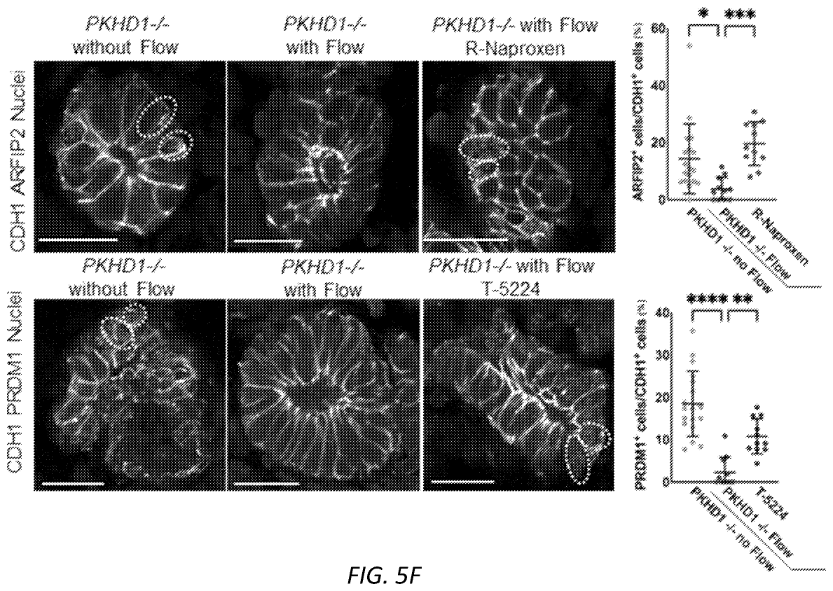

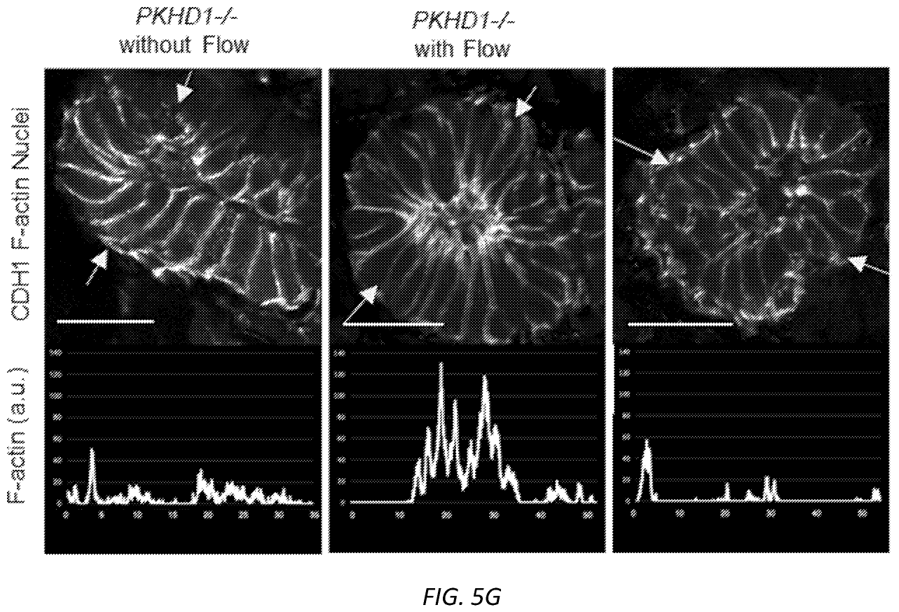

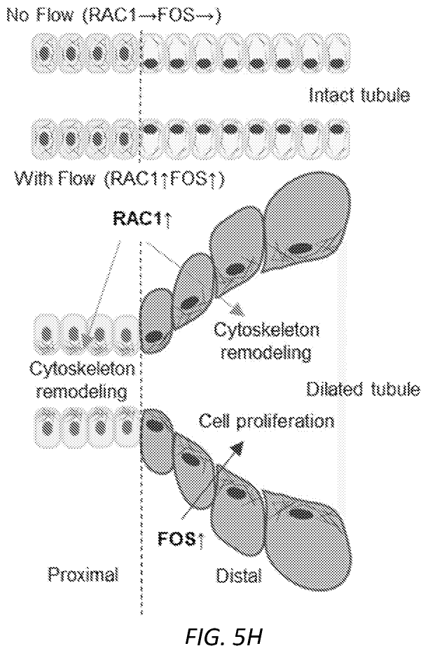

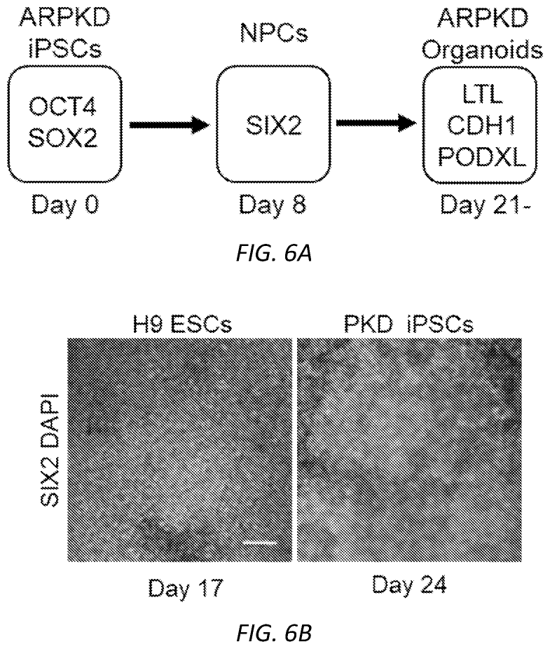

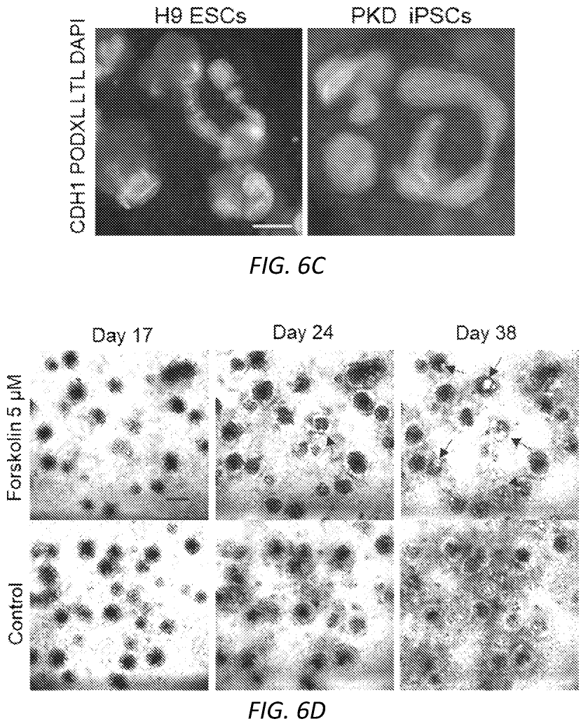

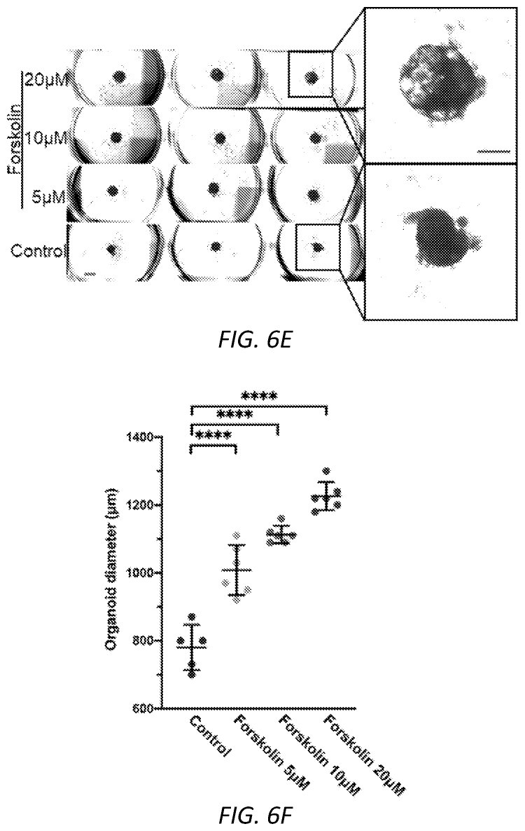

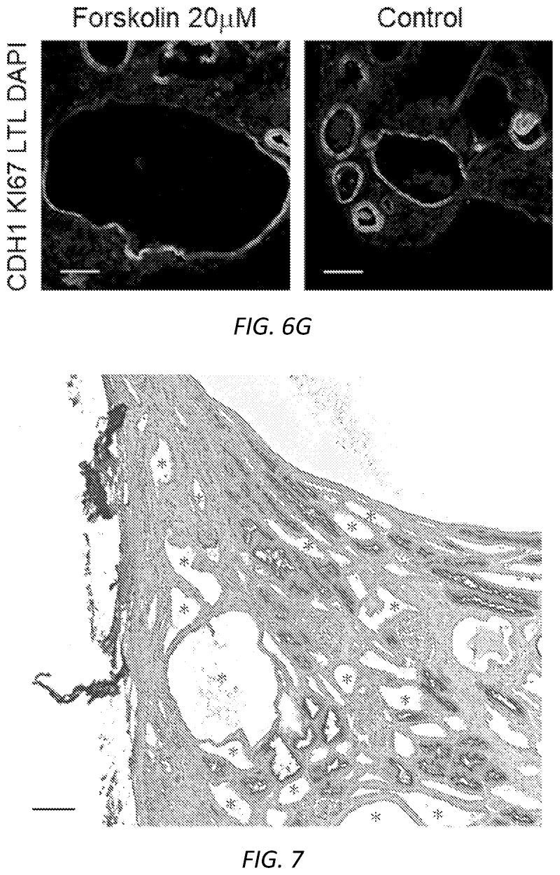

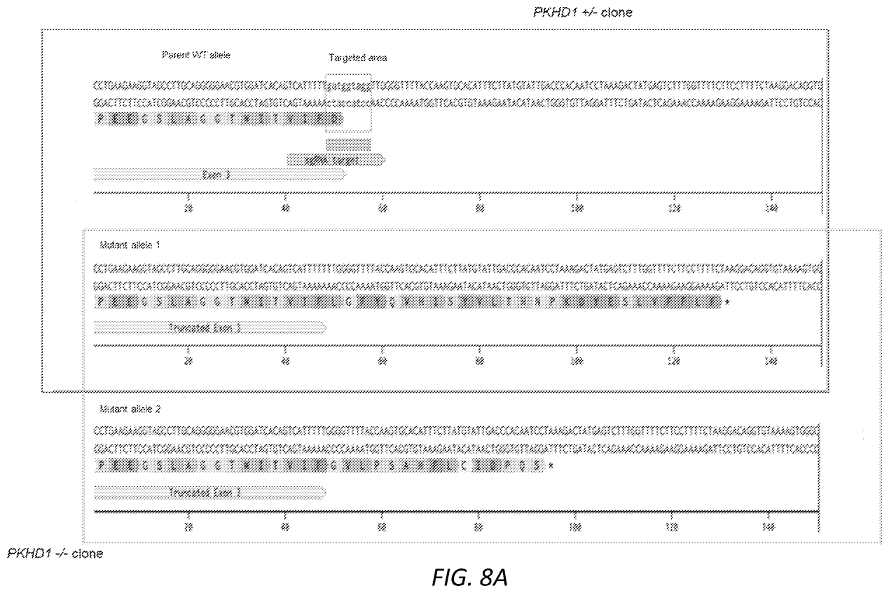

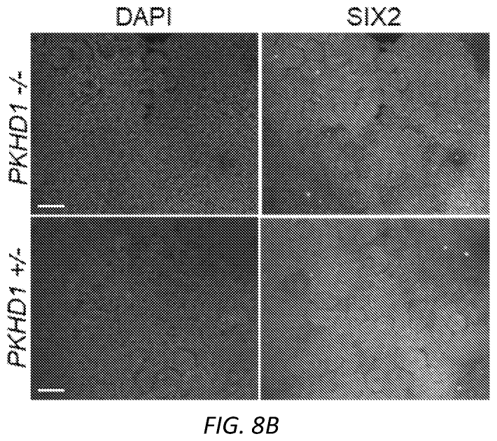

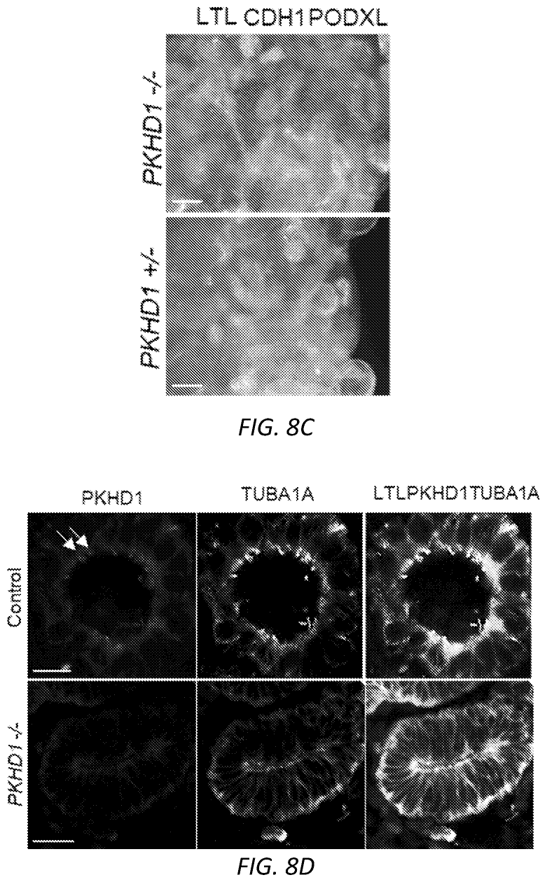

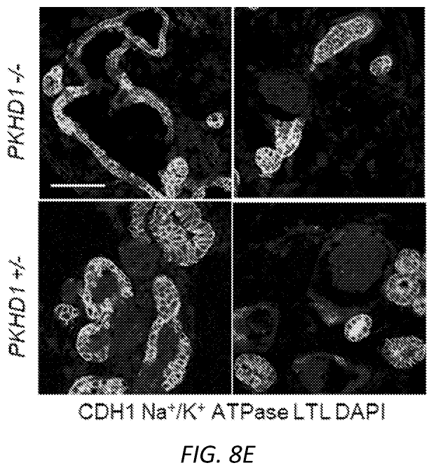

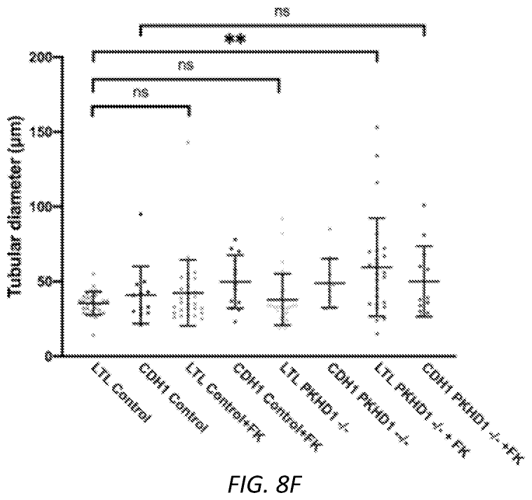

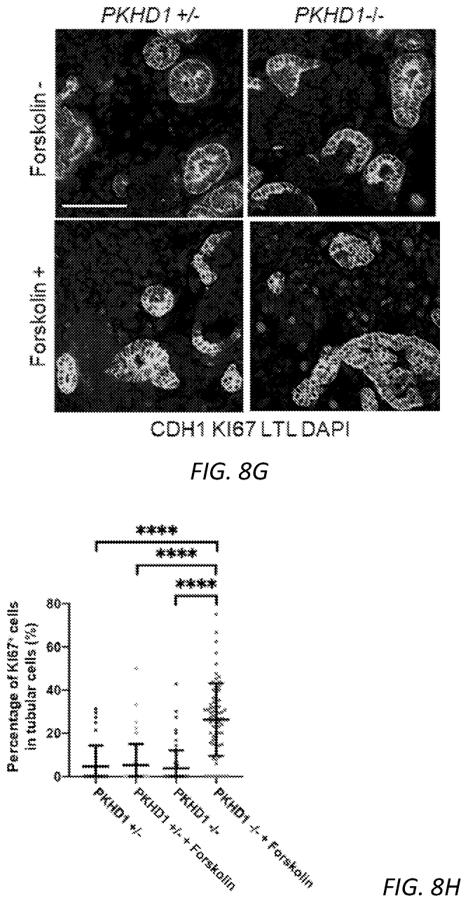

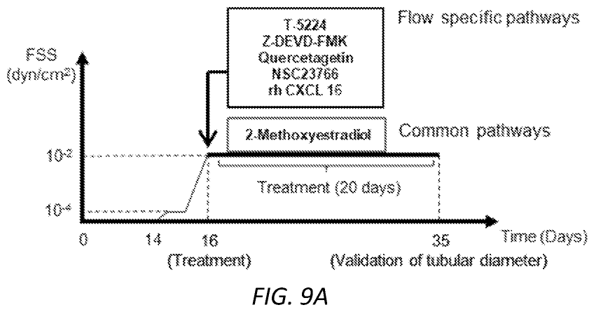

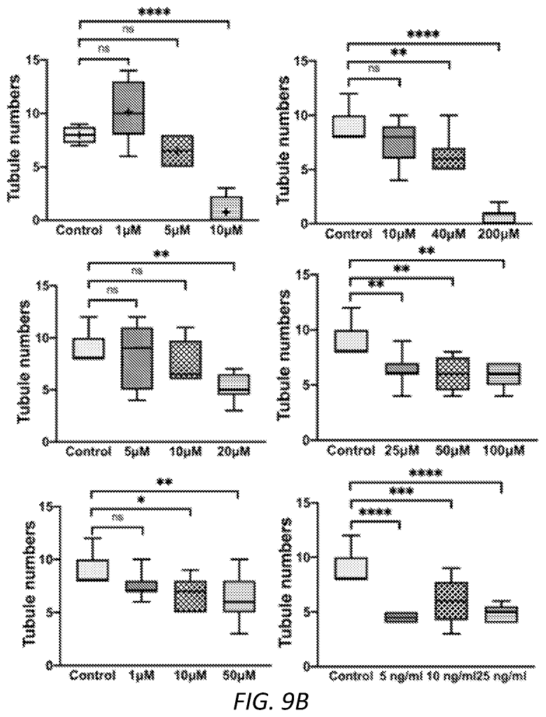

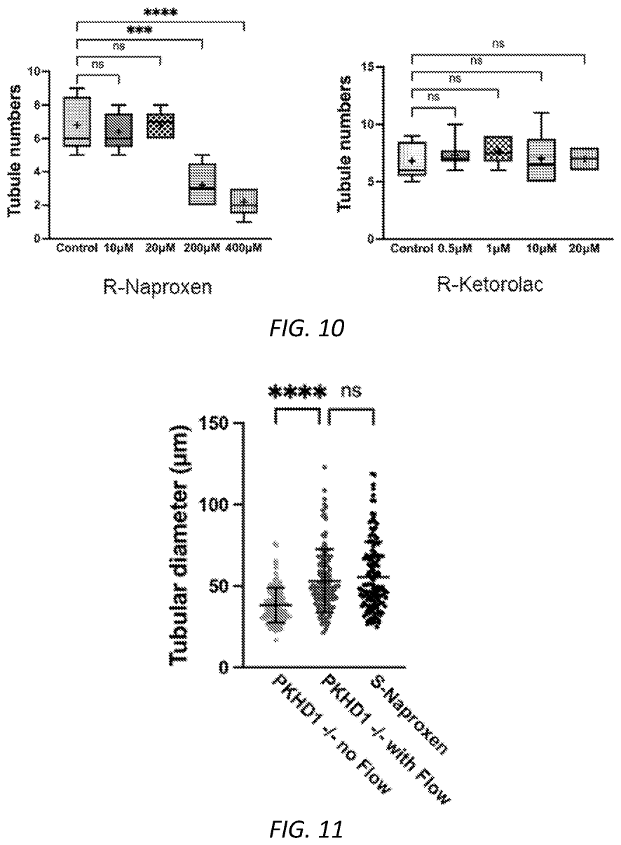

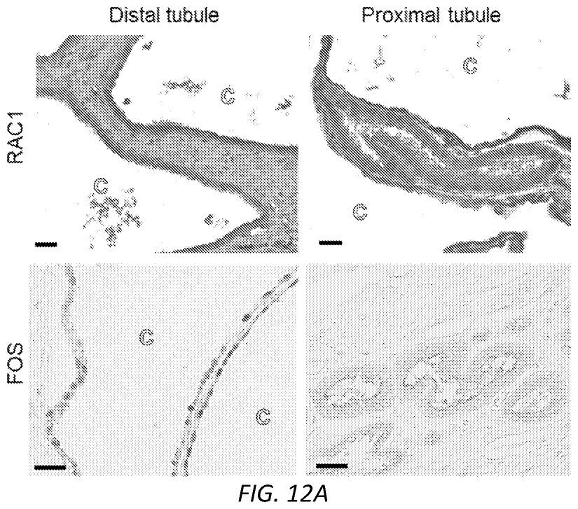

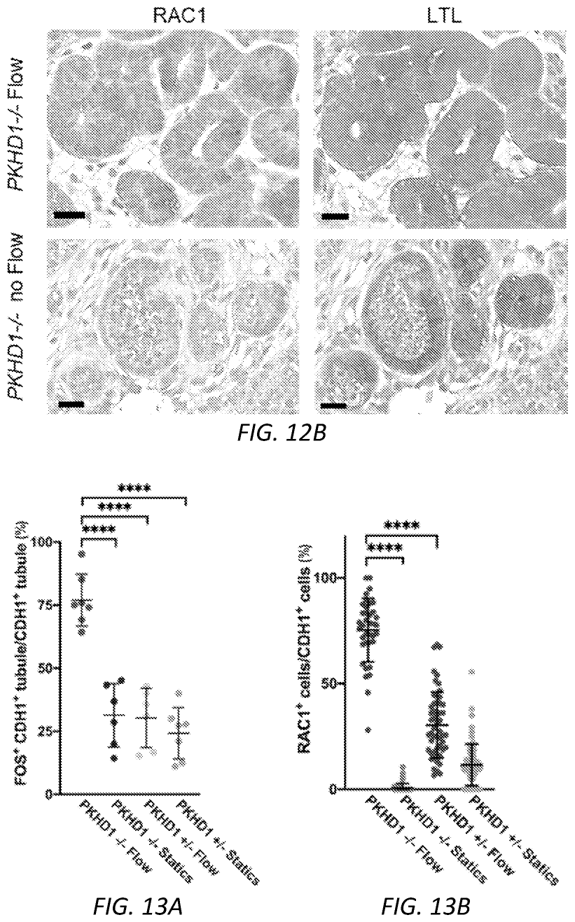

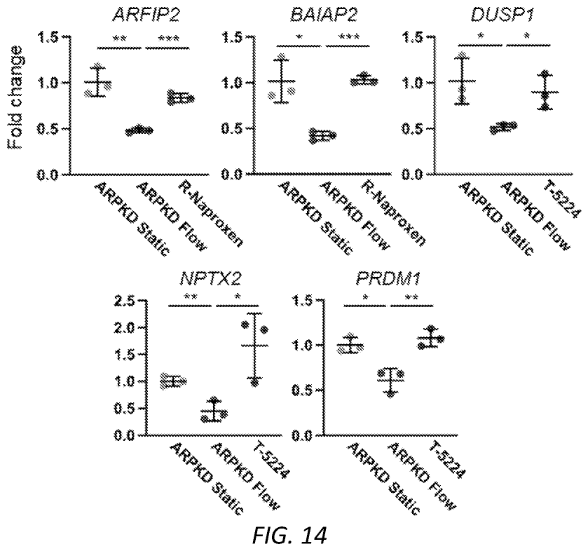

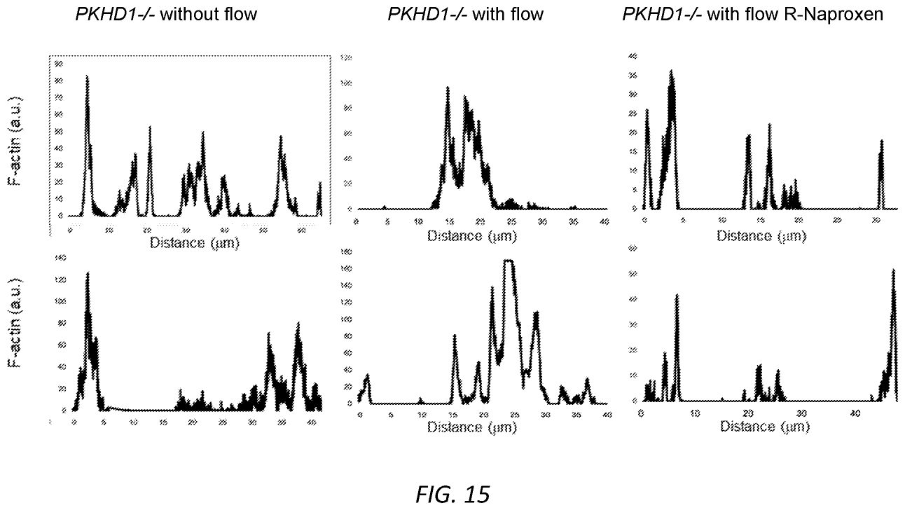

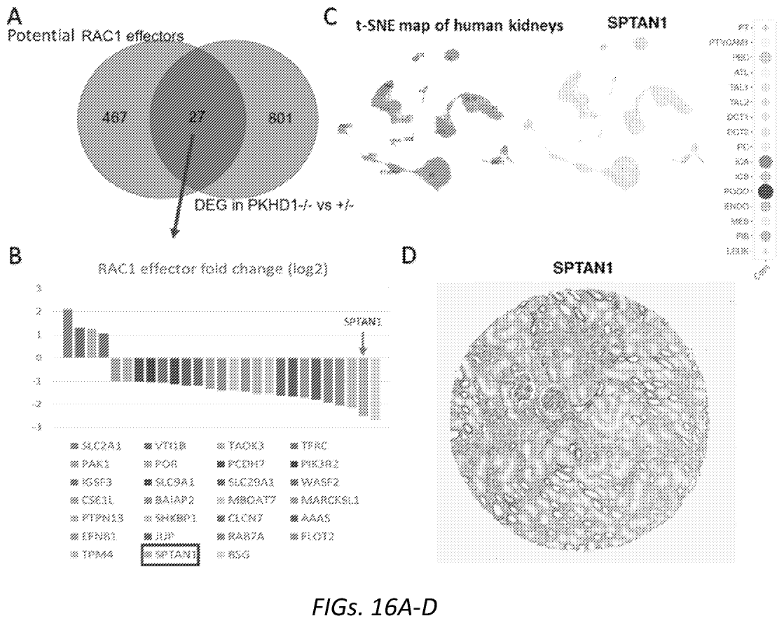

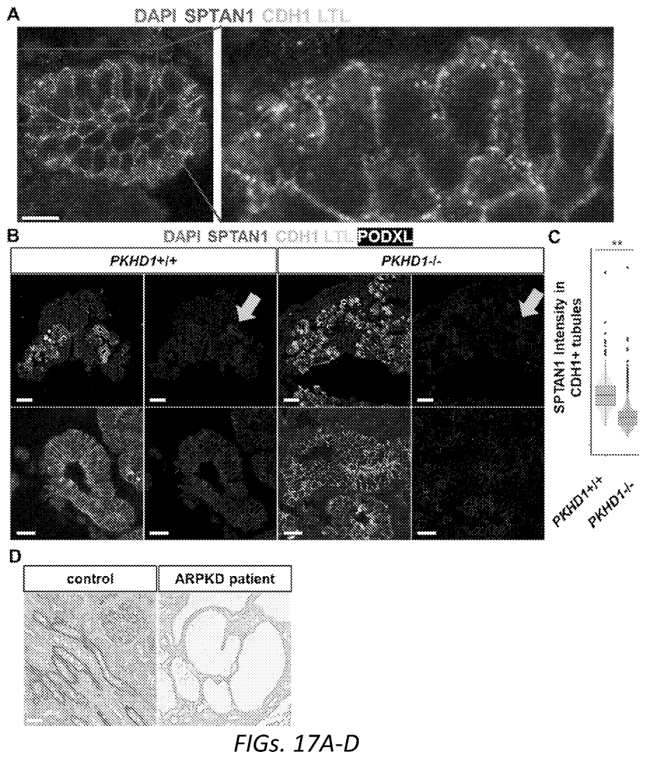

Data in all bar charts and dot plots are expressed as mean±s.d. Statistical analysis was done in GraphPad Prism 9, and statistical significance was attributed to values of P<0.05 as determined by one-way ANOVA with Tukey's multiple-comparisons test unless otherwise noted. Different significance levels (P values) are indicated in each figure with asterisks: *P<0.05, **P<0.01, ***P<0.001, and ****P<0.0001. For transparency, we state here the number of times experiments were repeated independently, with similar results obtained, to produce the data shown: FIG. 1D; 8 independent experiments were performed to confirm these phenotypes. FIG. 1F; n=74 LTL+ tubules with flow, n=66 LTL+ tubules in static culture, n=191 LTL+ tubules treated with forskolin, n=32 CDH1+ tubules with flow, n=27 CDH1+ tubules in static culture, and n=104 CDH1+ tubules treated with forskolin. FIG. 2C; n=16 tubules transfected with siControl, n=17 tubules transfected with siKIF3A. 8 organoids transfected with siControl, and 7 organoids transfected with siKIF3A from 2 independent chips were analyzed in FIG. 2C. FIG. 2E; n=145 tubules transfected with siControl, n=102 tubules transfected with siKIF3A. FIG. 2F; n=107 tubules transfected with siControl, n=58 tubules transfected with siKIF3A. 13 organoids transfected with siControl, and 12 organoids transfected with siKIF3A from 2 independent chips were analyzed in FIGS. 2E and 2F. FIG. 2J; n=4 (perfusion), n=4 (rest). FIG. 2K; n=3. FIG. 2L; 3 independent chips were analyzed for LMD perfusion experiment. FIG. 3E; n=141 tubules in PKHD1+/− organoids, n=181 tubules in PKHD1−/− organoids, n=289 tubules in PKHD1−/− organoids treated with T-5224, n=235 tubules in PKHD1−/− organoids treated with NSC23766, n=294 tubules in PKHD1−/− organoids treated with 2-MeOE₂, n=211 tubules in PKHD1−/− organoids treated with Z-DEVD-FMK, n=140 tubules in PKHD1−/− organoids treated with Quercetagetin, and n=150 tubules in PKHD1−/− organoids treated with rhCXCL16. 3 independent experiments were performed for FIG. 3E. FIG. 4C; n=90 tubules in PKHD1+/− organoids, n=122 tubules in PKHD1−/− organoids, n=76 tubules in PKHD1−/− organoids treated with R-Naproxen 20 μM, n=59 tubules in PKHD1−/− organoids treated with R-Ketorolac 1 μM, n=73 tubules in PKHD1−/− organoids treated with R-Ketorolac 10 μM, and n=76 tubules in PKHD1−/− organoids treated with T-5224. FIG. 4E; n=109 tubular clusters in PKHD1+/− organoids, n=139 tubular clusters in PKHD1−/− organoids, n=116 tubular clusters in PKHD1−/− organoids treated with R-Naproxen 20 μM, n=163 tubular clusters in PKHD1−/− organoids treated with R-Ketorolac 1 μM, n=171 tubular clusters in PKHD1−/− organoids treated with R-Ketorolac 10 μM, and n=83 tubular clusters in PKHD1−/− organoids treated with T-5224. FIG. 4F; n=48 tubular clusters in PKHD1+/− organoids, n=55 tubular clusters in PKHD1−/− organoids, n=48 tubular clusters in PKHD1−/− organoids treated with R-Naproxen 20 μM, n=48 tubular clusters in PKHD1−/− organoids treated with R-Ketorolac 1 μM, n=98 tubular clusters in PKHD1−/− organoids treated with R-Ketorolac 10 μM, and n=38 tubular clusters in PKHD1−/− organoids treated with T-5224. FIG. 4H; n=4 (control), n=4 (ARPKD). FIG. 4I; n=4 (control), n=4 (ARPKD). FIG. 5E; n=3 PKHD1−/− static organoids (no flow), n=3 PKHD1−/− organoids with flow condition, n=3 PKHD1−/− organoids treated with R-Naproxen 20 μM under flow condition, and n=3 PKHD1−/− organoids treated with T-5224 10 μM under flow condition were analyzed. FIG. 5F; n=18 CDH1⁺ tubules in PKHD1−/− static organoids (no flow), n=11 CDH1⁺ tubules in PKHD1−/− organoids with flow condition, and n=11 CDH1⁺ tubules in PKHD1−/− organoids treated with R-Naproxen 20 μM under flow condition were analyzed for ARFIP2 immunostaining. n=18 CDH1⁺ tubules in PKHD1−/− static organoids (no flow), n=11 CDH1⁺ tubules in PKHD1−/− organoids with flow condition, and n=10 PKHD1−/− organoids treated with T-5224 10 μM under flow condition were analyzed for PRDM1 immunostaining. FIG. 5G; n=3 CDH1⁺ tubules in PKHD1−/− static organoids (no flow), n=3 CDH1⁺ tubules in PKHD1−/− organoids with flow condition, and n=3 CDH1⁺ tubules in PKHD1−/− organoids treated with R-Naproxen 20 μM under flow condition. FIG. 6F; n=5 untreated organoids (control), n=6 organoids with forskolin 5 μM, n=6 organoids treated with forskolin 10 μM, and n=6 organoids treated with forskolin 20 μM. FIG. 8F; n=30 LTL tubules in control organoids without forskolin, n=12 CDH1⁺ tubules in control organoids without forskolin, n=29 LTL⁺ tubules in control organoids with FK, n=13 CDH1⁺ tubules in control organoids with FK, n=30 LTL⁺ tubules in PKHD1−/− organoids without FK, n=10 CDH1⁺ tubules in PKHD1−/− organoids without FK, n=26 LTL⁺ tubules in PKHD1−/− organoids with FK, and n=12 CDH1⁺ tubules in PKHD1−/− organoids with FK. FIG. 8H; n=35 tubules in PKHD1+/− organoids without forskolin, n=58 tubules in PKHD1+/− organoids with forskolin, n=66 tubules in PKHD1−/− organoids without forskolin, and n=69 in PKHD1−/− organoids with forskolin. FIG. 9B; n=7-8 untreated organoids (control), n=7 organoids with 2-MeOE₂ 1 μM, n=4 organoids with 2-MeOE₂ 5 μM, n=4 organoids with 2-MeOE₂ 10 μM, n=5 organoids with T-5224 5 μM, n=8 organoids with T-5224 10 μM, n=9 organoids with T-5224 20 μM, n=11 organoids with Z-DEVD-FMK 1 μM, n=14 organoids with Z-DEVD-FMK 10 μM, n=11 organoids with Z-DEVD-FMK 50 μM, n=11 organoids with Quercetagetin 10 μM, n=12 organoids with Quercetagetin 40 μM, n=11 organoids with Quercetagetin 200 μM, n=11 organoids with NSC23766 25 μM, n=9 organoids with NSC23766 50 μM, n=7 organoids with NSC23766 100 μM, n=8 organoids with rhCXCL16 5 ng/ml, n=8 organoids with rhCXCL16 10 ng/ml, and n=13 organoids with rhCXCL16 25 ng/ml. FIG. 10; n=5 untreated organoids (control), n=5 organoids with R-Naproxen 10 μM, n=5 organoids with R-Naproxen 20 μM, n=5 organoids with R-Naproxen 200 μM, n=5 organoids with R-Naproxen 400 μM, n=6 organoids with R-Ketorolac 0.5 μM, n=6 organoids with R-Ketorolac 1 μM, n=6 organoids with R-Ketorolac 10 μM, and n=5 organoids with R-Ketorolac 20 μM. FIG. 11; n=120 tubules in PKHD1−/− organoids with no flow, n=116 tubules in PKHD1−/− organoids with flow, and n=139 tubules in PKHD1−/− organoids treated with S-Naproxen 20 μM. FIG. 13A; n=7 organoids in PKHD1−/− organoids with flow condition, n=6 organoids in PKHD1−/− organoids without flow condition, n=6 organoids in PKHD1+/− organoids with flow condition, n=7 organoids in PKHD1+/− organoids without flow condition. FIG. 13B; n=37 tubules in PKHD1−/− organoids with flow condition, n=81 tubules in PKHD1−/− organoids without flow condition, n=51 tubules in PKHD1+/− organoids with flow condition, n=74 tubules in PKHD1+/− organoids without flow condition. FIG. 14; n=3 ARPKD patient-derived static organoids (no flow), n=3 ARPKD patient-derived organoids with flow condition, n=3 ARPKD patient-derived organoids treated with R-Naproxen 20 μM under flow condition, and n=3 ARPKD patient-derived organoids treated with T-5224 10 μM under flow condition were analyzed.

### Example 1. Fluidic Culture Induces Cysts in Distal Nephrons in PKHD1−/− Organoids

A prior ARPKD organoid model was developed using patient-derived iPS cells (17); however, cysts were not formed in static culture without cyclic adenosine monophosphate (cAMP) stimulation. When treated with cAMP inducers, forskolin or 8-Bromoadenosine 3′,5′-cyclic monophosphate (8-Br-cAMP), LTL⁺ proximal tubules formed cystic structures while CDH1⁺ distal nephrons rarely developed cysts. We also confirmed this non-physiological result using forskolin, which induced cyst formation in LTL⁺ proximal tubules in static organoids derived from ARPKD-patient iPSCs (FIG. 6A-G). However, ARPKD patients typically develop cysts in distal nephrons consisting of distal tubules and connecting tubules/collecting ducts (FIG. 7) (11), which arise in part due to flow-mediated mechanosensitive signaling (18). Hence, we posited that creating an ARPKD organoid-on-chip model that enables fluid flow akin to the native tissue microenvironment would yield clinically relevant phenotypes that recapitulate cystogenesis occurring in ARPKD patients.

To test the effects of flow-induced signals on cystogenesis in PKHD1-mutants, we utilized 3D-printed millifluidic chips (19). Kidney organoids at the renal vesicle stage on day 14 of differentiation were transferred onto a gelatin-fibrin (gelbrin) matrix contained within each chip and then subjected to fluid flow from day 14 to 35 (FIG. 1A, B) (1). For perfusion experiments, we used a parental human embryonic stem (ES) line, H9, and its CRISPR-mutant lines with PKHD1 hetero/homozygous mutations to correlate genotypes with phenotypes (FIG. 8A-D). Cyst-like structures formed and gradually enlarged over time in PKHD1−/− organoids, whereas PKHD1+/− organoids did not exhibit such structures in the same fluidic culture (FIG. 1C). For unbiased quantitative assessment of tubular enlargement, z-stack images were acquired from the top to bottom of each organoid after whole-mount immunostaining for LTL and CDH1 with sample clearing in flow or forskolin treated PKHD1−/− organoids (FIG. 1D, E). Short diameters of each proximal tubule (LTL⁺ CDH⁻) and distal nephron (LTL⁻ CDH⁺) were measured. We found that the proximal tubular diameter in PKHD1−/− organoids subjected to fluid flow remained unchanged, while the distal nephron diameter increased from 33.9±12.0 μm to 49.8±17.9 μm (p=0.0001), consistent with segment-specificity in ARPKD patients (FIG. 1F left). By contrast, forskolin increases the diameter in both LTL⁺ CDH⁻ and LTL⁻ CDH1⁺ tubules (FIG. 1F right, FIG. 8E-H). To further validate whether our organoid-on-chip model recapitulates human ARPKD, we evaluated cAMP and mammalian target of rapamycin (mTOR) signaling, both of which have been implicated in ARPKD cystogenesis (20, 21). cAMP levels in PKHD1−/− organoids were significantly higher in organoids subjected to flow compared to those maintained under static conditions. Flow also activated the mTOR pathway, as assessed by increased phospho-S6 Ribosomal Protein (pS6RP) in PKHD1−/− organoid compared to PKHD1+/−. These findings suggest that the flow-induced ARPKD model recapitulated the requisite pathogenesis to enable identification of therapeutic targets.

### Example 2. Mechanosensing Signals are Activated by Fluid Flow

Conflicting data exist on whether ciliary signals facilitate cystogenesis in ARPKD patients (22, 23). Loss of cilia suppressed renal cyst growth in rodent ADPKD models (24, 25). By contrast, disruption of cilia in Pkhd1-mutant mice did not suppress liver cyst formation or progression, calling into question whether primary cilia are implicated in ARPKD pathogenesis (26). To determine whether ciliary stress is involved in cyst formation in our model, we carried out microarray analysis using 3D-Gene® Human Oligo chips. Control organoids derived from PKHD1+/− mutants were subjected to fluid flow on chip from day 14 of differentiation and harvested on day 35. Direct comparison between flow and static culture conditions in control organoids revealed more mature nephron structures developed under flow, as demonstrated by a significant enrichment of hallmark gene sets for each segment, including proximal tubules, distal tubules, and cilia. Differential expression gene (DEG) analysis identified 655 upregulated and 225 downregulated genes compared to the static control (FIG. 2A, upper). Genes upregulated by flow were implicated in 111 gene ontology (GO) terms that are involved in cilia-related signals, including calcium and cAMP signals (FIG. 2A, lower) (27), suggesting genes associated with the ciliary signals were activated in our kidney organoids-on-chip model.

Next, we used siRNA to knock-down primary cilia by targeting KIF3A that encodes one of the intraflagellar transports (IFTs) (24). siKIF3A was transfected twice on day 8 and 16 of differentiation, and PKHD1−/− organoids were subjected to flow from day 14 until the samples are harvested on day 22. A significant reduction of ciliated tubular cells from 81.0±14.0% to 14.1±10.4% was confirmed by z-stack imaging for acetylated α-tubulin (TUBA1A) (FIG. 2B, C). While dramatic cyst formation was not observed in both siKIF3A and scrambled siRNA (siControl) samples at this early stage on day 22, ciliary knockdown significantly reduced KI67⁺ proliferating tubular cells from 18.5±15.9% to 3.8±5.0% in PKHD1−/− organoids with a significant reduction of tubular diameter compared to siControl under flow (FIGS. 2D, E, F). Together with GO term analysis, these results suggest the involvement of ciliary stress in cystogenesis in ARPKD organoids cultured under flow.

While the primary cilia serve as key mechanosensors, other cellular mechanosensing mechanisms such as the cytoskeleton-nucleus axis are also involved in the conversion of physical forces into biochemical and electrical signals for cellular responses (28). Indeed, we found that fluid flow activated 168 GO terms related to mechanosensing signals in kidney organoids (FIG. 2G), including 61 GO terms related to cell adhesion, actin cytoskeleton, and membrane tension (29). Because flow was applied across whole kidney organoids in our model, we hypothesized that the induced mechanical stimuli might lead to a change in membrane tension within the organoids (30).

To determine whether cellular membrane tension is altered by mechanical forces in the kidney organoids-on-chip model, we used the fluorescent lipid tension reporter (FliptR) to visualize plasma membranes in live cells via fluorescent intensity. Simultaneously, membrane tension changes can be detected by fluorescence lifetime imaging microscopy (FLIM), where longer lifetimes (τ₂) of the FliptR dye signify higher tension (31). Wild-type (WT) organoids cultured under flow were stained with FliptR and a live nuclear dye between days 16-18. Tubular nuclei and membranes were then imaged in live organoids using confocal microscopy, which revealed oscillatory motion of tubular basement membranes under fluidic shear stress (FSS) (FIG. 2H) (19, 32). The FliptR lifetimes were captured first under superfusive flow, then after 1 h of static conditions in the same tubular structures. The range of longer lifetime changes (Δτ₂) in perfusion condition (0.89±0.07 ns) was broader than in static condition (0.47±0.08 ns), while the fluorescent intensity of FliptR was unchanged (FIG. 2I, J). Notably, the overall lifetime distribution in cellular membranes was increased from 2.67±1.81 ns to 3.17±1.38 ns during flow, whereas it did not change during the rest period (2.77±1.68 ns to 2.86±1.65 ns) (FIG. 2K). These data suggest that fluidic culture induces cellular stretching in organoid tubules, which may provide more physiological platforms for ARPKD modeling than those cultured under static conditions (33).

Next, we evaluated luminal flow by time-lapse live imaging of organoids-on-chip after live staining for fluorescently labeled wheat germ agglutinin (WGA) that binds to tubular epithelial membranes. Fluorescently labeled low-molecular-weight dextran (LMD) was then slowly perfused by gravity to minimize the motion of tubules during live imaging. We initially observed LMD signals in the glomerular-like structures in 11 minutes of perfusion that were subsequently detected in the tubular lumens within 15 minutes after perfusion was initiated (FIG. 2L). These results suggest the presence of tubular luminal flow that activates mechanosensing in organoid tubules together with the interstitial flow. Although determining the influx route into the tubular lumens would require further study using reporter lines and high-resolution live imaging, we speculate that luminal flow may be initiated from glomerular vascular poles as LMD was first detected in those regions. We suspect that the glomerular vascular clefts may not fully closed at this early developmental stage. Given their flow-induced tubular enlargement, we posit that mechanosensing signals induced by flow might be essential for ARPKD pathogenesis.

### Example 3. Identification of Novel Therapeutic Targets for ARPKD

To investigate cystogenic signals, we compared the global gene expression of PKHD1−/− organoids to PKHD1+/− in each of flow, forskolin, or static conditions independently (FIG. 3A upper). DEG analysis between PKHD1−/− and +/− identified 407 signals altered in the flow condition, 63 signals in forskolin, and 72 signals under static culture (FIG. 3A lower). Since static organoids did not exhibit cysts in PKHD1−/−, we disregarded the 72 non-cystogenic signal alterations seen in static culture. While 32 pathways were involved in both flow and forskolin conditions, 353 signal pathways were specific to organoids cultured under flow (FIG. 3A lower). Following a comprehensive literature search, we excluded 124 previously reported cytogenic signals identified in AD/ARPKD studies (20, 27, 34, 35). Thus, we have identified 229 unreported pathways. We then categorized all signal pathways into biological processes for visualization (FIG. 3B), namely cytoskeleton remodeling, immune response, GPCR-related, cell proliferation, growth factor, erythropoietin, hormone, cell adhesion, and others. 124 reported signals, including cAMP, WNT, TNF alpha, and ERK-MAPK, validate the use of our kidney organoids-on-chip platform for ARPKD modeling.

Five target molecules from 229 flow-specific signals and 1 target from 32 common signals were selected for further evaluation (FIG. 3C upper). The selected compounds and target molecules were T-5224 for FOS/Activator protein 1 (AP-1), Z-DEVD-FMK for caspase, Quercetagetin for BCL2 associated agonist of cell death (BAD), NSC23766 for Rac Family Small GTPase 1 (RAC1), recombinant human C—X—C Motif Chemokine Ligand 16 (rhCXCL16) for CXCL16 (FIG. 3C lower red), and 2-Methoxyestradiol (2-MeOE₂) for Hypoxia-Inducible Factor-1 (HIF-1) (FIG. 3C lower gray). PKHD1−/− organoids were treated with those compounds from day 16 to 35 of differentiation in fluidic culture (FIG. 9A). The optimal concentrations of all compounds were determined within the range used in previous studies by evaluating normal nephron formation in static culture (FIG. 9B). Unbiased 3D imaging by tissue clearing of whole organoids revealed a significant reduction of CDH1⁺ tubular enlargement during treatment with T-5224, NSC23766, and 2-MeOE₂ (FIG. 3D, E). By contrast, Z-DEVD-FMK showed a marginal effect, while Quercetagetin and rhCXCL16 did not affect cyst suppression. Closer examination of phenotypes, and associated molecular genetics, revealed convergent and divergent pathogenic mechanisms between ADPKD and ARPKD (27). To further investigate the utility of these candidates in ADPKD, we carried out global gene expression profiling comparing our ARPKD datasets with a meta-analysis-based dataset of human, rat, and mouse ADPKD tissues (36, 37). RAC1- and FOS-related mechanosensory signals were unchanged in large cysts when compared to non-cystic tissue in human ADPKD (FIG. 3F) (38, 39). This may have an anatomical explanation as urinary flow is absent in large ADPKD cysts that are non-continuous with the tubular lumen, whereas ARPKD cysts are tubular dilatations (11).

### Example 4. Potential Clinical Translation from Existing Drugs for ARPKD

Validation is a critical step during drug-discovery, since most candidate drugs are deemed ineffective or toxic (40). To mitigate safety concerns, we explored existing drugs that inhibit RAC1, FOS, or HIF-1 as potential new therapeutic targets. Of these, HIF-1 inhibitor is excluded from candidates, since HIF-1 suppression exacerbates renal anemia via impaired erythropoietin production (41). We focused on R-Naproxen and R-Ketorolac, two widely-used FDA-approved nonsteroidal anti-inflammatory drugs (NSAIDs) that also have an inhibitory effect against RAC1 (42, 43), as well as a selective FOS inhibitor, T-5224 (44), chosen for favorable safety profile during clinical trials for arthritis (45) (FIG. 4A).

PKHD1−/− organoids-on-a-chip were treated with these compounds from day 16 to 35 of differentiation in fluidic culture. The optimal concentration of each compound was determined based on previous studies and their negligible impact on nephron formation (FIG. 10A) (46). Nephrotoxicity of each chemical compound was assessed by kidney injury molecule-1 (KIM-1) and double-stranded DNA breaks (DSBs) marked by γH2A histone family member X (γH2AX). Notably, none of these compounds resulted in any signs of toxicity to nephron epithelial and stromal cells at the concentration tested for cyst suppression. 3D imaging revealed significant reductions of CDH1⁺ tubular enlargement upon treatment by R-Naproxen, R-Ketorolac, and T-5224 (FIG. 4B, C). To validate their therapeutic effects, distal tubular volume and KI67⁺ percentage in distal nephrons were measured by image analysis (Imaris) and shown to be significantly improved by these candidates (FIG. 4D, E, F). To exclude the possibility of another therapeutic mechanism by an anti-inflammatory effect of R-Naproxen, organoids were treated with S-Naproxen, an optical isomer of R-Naproxen, that lacks RAC1 inhibitory activity but has an anti-inflammatory effect (47). 3D imaging confirmed S-Naproxen did not inhibit cyst formation in PKHD1−/− organoids cultured under flow, suggesting the cyst suppression was not mediated by the anti-inflammatory effect of R-Naproxen (FIG. 11).

To determine whether RAC1 and FOS were activated in ARPKD patients, we immunostained for Rac1-guanosine triphosphate (Rac1-GTP; an activated form of RAC1) and FOS in kidney samples from four ARPKD patients and normal portions of tumor nephrectomy as a control. In human ARPKD samples, RAC1⁺ cells and FOS' cells were 94.8±3.5% and 50.9±6.2% respectively in cystic epithelial cells, significantly higher than the controls with 11.1±1.7% and 15.1±6.6% positivity in distal tubules (FIG. 4G, H, I). Of note, while RAC1⁺ cells were also observed in proximal tubules, FOS' proximal tubules were rarely detected in ARPKD patients (FIG. 12A). The increased expression of RAC1 in proximal and distal nephrons and FOS in enlarged distal nephrons was also confirmed in PKHD1−/− organoids cultured under flow (FIG. 12B, FIG. 13A, B).

Based on our observation of FOS activation in cystic epithelial cells in ARPKD patient kidneys, we postulated that FOS may be a crucial determinant of the cyst origins. To investigate the cell type-specific FOS expression in human kidneys, we analyzed single cell RNA-seq, regional transcriptomics, and assayed for transposase-accessible chromatin using sequencing (ATAC-seq) from the Kidney Precision Medicine Project (KPMP) and the Kidney Interactive Transcriptomic (KIT) (48). As expected from our immunostaining results in human ARPKD tissues (FIG. 12A), FOS expression was predominantly detected in distal nephrons and only sparsely found in proximal tubules, which supports our observation (49). Conversely, mouse kidney datasets of single cell RNA-seq from the KIT showed FOS expression in both proximal tubules and collecting ducts in mice (50), consistent with cyst formation in the S3 segment of proximal tubules in mouse ARPKD model (12), and in proximal tubules and collecting ducts in PCK mouse (51). Given the FOS expression in specific tubular segments where cysts develop in human and mouse ARPKD, FOS might be an important molecule that determines segment specificity of cystogenesis in ARPKD.

### Example 5. RAC1 and FOS Involvement in ARPKD Cystogenesis

Prior studies demonstrated that mechanical stress activates RAC1 and FOS by stimulating basal adhesion proteins such as integrins involved in the activation of the Rho family of GTPase proteins including RAC1 (39, 52). FOS is one of downstream molecules of RAC1, regulating cell proliferation (53). To further investigate the cystogenic mechanisms initiated by mechanical stress in our ARPKD model, we analyzed gene expression involved in RAC1 and FOS pathways defined by MetaCore. Interestingly, many downstream genes of RAC1 and FOS were downregulated by flow in PKHD1−/− organoids, while flow activates those genes in the PKHD1+/− control (FIG. 5A, S16A, B). Among these genes, ARFIP2, BAIAP2, WASF2, ACTB, and ACTG2 are implicated in “cytoskeleton remodeling” under RAC1 signal according to MetaCore, and CAMP, DUSP1, NPTX2, PRDM1, and SOCS3 are involved in “negative regulation of cell proliferation”, that downregulate cell cycle, in FOS pathway (FIG. 5B) (54-58). In contrast, the expression of other RAC1 downstream molecules was not altered by PKHD1−/− mutations, that include NCF1 and PRD6B involved in “reactive oxygen species production” and “establishment of cell polarity” respectively (FIG. 5B) (59, 60). Given that the inhibitors of RAC1 or FOS ameliorated cystogenesis in our ARPKD model, these results suggest that PKHD1−/− mutations conversely downregulate cytoskeleton genes and negative cell cycle regulators upon activation of RAC1 and FOS and lead to cyst formation.

To validate whether “cytoskeleton remodeling” and “negative regulation of cell proliferation” are crucial for cystogenesis and contrarily regulated under RAC1 and FOS in PKHD1−/− mutants, we referred to another genome informatics database (available at informatics.jax.org). Genes associated with these biological processes were selected by GO terms namely “actin cytoskeleton” and “negative regulation of cell population proliferation”. The heat map of these genes revealed a reduction of actin cytoskeleton gene expression in PKHD1−/− organoids compared to PKHD1+/− organoids when cultured under flow (FIG. 5C, right), consistent with actin cytoskeleton reduction in PKD1-deficient mouse kidneys (61, 62). Furthermore, the expression of genes related to negative regulation of cell proliferation was substantially reduced in PKHD1−/− organoids (FIG. 5C, left). These results validate that PKHD1−/− mutations result in converse regulation of genes associated with cytoskeleton and negative cell cycle regulation when stimulated by flow.

To understand the therapeutic mechanisms of R-Naproxen and T-5224 via modulating RAC1 and FOS signals, PKHD1−/− organoids and ARPKD patient-derived organoids cultured under flow were treated with these drugs from day 16 of differentiation and harvested on day 23 (FIG. 5D). Quantitative polymerase chain reaction (qPCR) confirmed flow-induced downregulation of RAC1-downstream genes, ARFIP2 and BAIAP2, in both CRISPR-mutant and patient-derived organoids. As expected, R-Naproxen rescued the expression of these two genes (FIG. 5E, 14). Similarly, qPCR of the FOS-downstream genes, DUSP1, NPTX2, and PRDM1, demonstrated their downregulation by flow and reverse expression by T-5224. The quantitative immunohistochemistry for ARFIP2, and PRDM1, the downstream genes of RAC1 and FOS respectively, validated their decreased expression by flow and the rescue by R-Naproxen and T-5224 (FIG. 5F).

To discern how flow and PKHD1−/− mutations affect cytoskeleton remodeling in CDH1⁺ organoid tubules, we examined F-actin localization (63). PKHD1−/− organoids under flow conditions were treated with R-Naproxen for 7 days from day 16 and assessed by phalloidin staining. In PKHD1−/− organoids without flow, the actin fibers were widely distributed throughout the cytoplasm in CDH1⁺ tubules (FIG. 5G, left, FIG. 15), whereas PKHD1−/− organoids under flow showed basal diminution and apical enrichment of actin fibers (FIG. 5G, middle, FIG. 15). In control organoids derived from the parental hPSC line, such redistribution of actin fibers was not observed under flow. The apical redistribution of actin fiber was ameliorated by R-Naproxen treatment in PKHD1−/− mutants (FIG. 5G, right, FIG. 15), collectively suggesting cytoskeleton remodeling observed in PKHD1−/− mutants is a disease phenotype and can be reversed by R-Naproxen. Interestingly, this cytoskeleton remodeling was also observed in proximal tubules in PKHD1−/− organoids under flow conditions, which was not detected in static PKHD1−/− organoids nor control organoids under flow. Taken together, RAC1 and FOS activation initiated by mechanical stress (38, 64, 65) induce cytoskeleton remodeling and cell proliferation in distal nephrons, which can be potentially treated by R-Naproxen and T-5224 in ARPKD patients (FIG. 5H).

### Example 6. RAC1 Effector Genes are Downregulated in PKHD1−/− Mutants

The Rho family of GTPases including RAC1 is known to take actions with effector proteins, and RAC1 downstream processes can be changed by the effectors (80). To assess the RAC1 effector expression, we used gene lists of potential effectors of major Rho GTPases, recently published by Bagci et al (81). They used HEK293, a human fetal kidney cell line, to evaluate the proximity interaction of Rho GTPases including RAC1. We chose the gene list of the RAC1 potential effectors and compared them to DEGs between PKHD1−/− and +/− mutant organoids (FIG. 16A). Interestingly, 27 RAC1 effectors were significantly altered in PKHD1−/− mutants, and the majority of those were downregulated (FIG. 16B). Hence, the overexpression of active RAC1 in cystic epithelia might be explained by the reduced consumption of active RAC1 due to the downregulation of RAC1 effector proteins. Then, we looked at each gene of the 27 effectors in literature and the human kidney database and found that SPTAN1 could be the major mechanism of actin-cytoskeleton dysregulation observed in the ARPKD organoids. SPTAN1 is expressed in tubular epithelial cells predominantly in collecting ducts at mRNA levels but also in other tubular segments at protein levels in normal human kidneys (FIG. 16C, D). These data suggest that RAC1 maintains normal cellular architecture via SPTAN1 in the normal condition, and PKHD1 mutations cause the reduction of SPTAN1 and shift the RAC1 downstream signals to pathological cellular remodeling towards cyst formation.

### Example 7. RAC1 Effector, SPTAN1 is Decreased in Cystic Epithelia in Both ARPKD Organoids and Patient Kidneys

To determine if the RAC1 effector SPTAN1 is decreased in ARPKD, we conducted additional experiments to evaluate the expression of SPTAN1, a filamentous cytoskeletal protein. We performed immunostaining for SPTAN1 in kidney organoids cultured under flow conditions on perfusion chips during days 22-24 of differentiation, earlier time points before cystic structures become apparent. In wild-type (PKHD1+/+) organoids cultured under flow, we observed strong expression of SPTAN1 in CDH1⁺ tubules. The localization of SPTAN1 in the cytoplasm under the cell membranes was consistent with the known localization of cytoskeletal proteins (FIG. 17A). In line with the RNA results, the expression of SPTAN1 was decreased in PKHD1−/− ARPKD organoids cultured under flow (FIG. 17B). To quantitatively assess this downregulation, we utilized Imaris software to measure the intensities of SPTAN1 in each CDH1-positive cellular object. Our analysis confirmed the statistical significance of SPTAN1 downregulation in ARPKD organoids (FIG. 17C). Furthermore, we examined the expression of SPTAN1 in human kidney tissue using immunohistochemistry with the same primary antibody. In normal kidney controls, SPTAN1 was predominantly expressed in distal nephrons. However, in ARPKD patients, we observed suppression of SPTAN1 in the cystic epithelium (FIG. 17D), which is consistent with the phenotypes observed in the ARPKD organoids. These results collectively support our proposed mechanism that the reduced effector protein of RAC1, SPTAN1, plays a facilitating role in cyst formation.

### REFERENCES

1. R. Morizane, A. Q. Lam, B. S. Freedman, S. Kishi, M. T. Valerius, J. V. Bonventre, Nephron organoids derived from human pluripotent stem cells model kidney development and injury. Nature biotechnology 33, 1193-1200 (2015).
2. R. A. Wimmer, A. Leopoldi, M. Aichinger, N. Wick, B. Hantusch, M. Novatchkova, J. Taubenschmid, M. Hammerle, C. Esk, J. A. Bagley, D. Lindenhofer, G. Chen, M. Boehm, C. Agu, F. Yang, B. Fu, J. Zuber, J. Knoblich, D. Kerjaschki, J. Penninger, Human blood vessel organoids as a model of diabetic vasculopathy. Nature 565, 505-510 (2019).
3. H. Koike, K. Iwasawa, R. Ouchi, M. Maezawa, K. Giesbrecht, N. Saiki, A. Ferguson, M. Kimura, W. L. Thompson, J. M. Wells, A. M. Zorn, T. Takebe, Modelling human hepato-biliary-pancreatic organogenesis from the foregut-midgut boundary. Nature 574, 112-116 (2019).
4. K. Fujimori, M. Ishikawa, A. Otomo, N. Atsuta, R. Nakamura, T. Akiyama, S. Hadano, M. Aoki, H. Saya, G. Sobue, H. Okano, Modeling sporadic ALS in iPSC-derived motor neurons identifies a potential therapeutic agent. Nature medicine 24, 1579-1589 (2018).
5. M. Dvela-Levitt, M. Kost-Alimova, M. Emani, E. Kohnert, R. Thompson, E.-H. Sidhom, A. Rivadeneira, N. Sahakian, J. Roignot, G. Papagregoriou, M. S. Montesinos, A. R. Clark, D. McKinney, J. Gutierrez, M. Roth, L. Ronco, E. Elonga, T. A. Carter, A. Gnirke, M. Melanson, K. Hartland, N. Wieder, J. C. H. Hsu, C. Deltas, R. Hughey, A. J. Bleyer, S. Kmoch, M. Zivna, V. Baresova, S. Kota, J. Schlondorff, M. Heiman, S. L. Alper, F. Wagner, A. Weins, T. R. Golub, E. S. Lander, A. Greka, Small molecule targets TMED9 and promotes lysosomal degradation to reverse proteinopathy. Cell 178, 521-535. e523 (2019).
6. N. Gupta, T. Matsumoto, K. Hiratsuka, E. G. Saiz, P. Galichon, T. Miyoshi, K. Susa, N. Tatsumoto, M. Yamashita, R. Morizane, Modeling injury and repair in kidney organoids reveals that homologous recombination governs tubular intrinsic repair. Science Translational Medicine 14, eabj4772 (2022).
7. L. F. Menezes, Y. Cai, Y. Nagasawa, A. M. Silva, M. L. Watkins, A. M. Da Silva, S. Somlo, L. M. Guay-Woodford, G. G. Germino, L. F. Onuchic, Polyductin, the PKHD1 gene product, comprises isoforms expressed in plasma membrane, primary cilium, and cytoplasm. Kidney international 66, 1345-1355 (2004).
8. L. M. Guay-Woodford, R. A. Desmond, Autosomal recessive polycystic kidney disease: the clinical experience in North America. Pediatrics 111, 1072-1080 (2003).
9. E. A. Hartung, L. M. Guay-Woodford, Autosomal recessive polycystic kidney disease: a hepatorenal fibrocystic disorder with pleiotropic effects. Pediatrics 134, e833-e845 (2014).
10. K. Burgmaier, S. Kilian, B. Bammens, T. Benzing, H. Billing, A. Buscher, M. Galiano, F. Grundmann, G. Klaus, D. Mekahli, L. Michel-Calemard, G. Milosevski-Lomic, B. Ranchin, K. Sauerstein, S. Schaefer, R. Shroff, R. Sterenborg, S. Verbeeck, L. T. Weber, D. Wicher, E. Wühl, J. Dotsch, F. Schaefer, M. C. Liebau, Clinical courses and complications of young adults with autosomal recessive polycystic kidney disease (ARPKD). Scientific reports 9, 1-11 (2019).
11. C. Bergmann, L. M. Guay-Woodford, P. C. Harris, S. Horie, D. J. Peters, V. E. Torres, Polycystic kidney disease. Nature reviews Disease primers 4, 1-24 (2018).
12. J. Woollard, R. Punyashtiti, S. Richardson, T. Masyuk, S. Whelan, B. Huang, D. Lager, V. Torres, V. Gattone, N. LaRusso, P. C. Harris, C. J. Ward, A mouse model of autosomal recessive polycystic kidney disease with biliary duct and proximal tubule dilatation. Kidney international 72, 328-336 (2007).
13. S. B. Mason, Y. Liang, R. M. Sinders, C. A. Miller, T. Eggleston-Gulyas, R. Crisler-Roberts, P. C. Harris, V. H. Gattone, Disease stage characterization of hepatorenal fibrocystic pathology in the PCK rat model of ARPKD. The Anatomical Record: Advances in Integrative Anatomy and Evolutionary Biology 293, 1279-1288 (2010).
14. K.-J. Jang, A. P. Mehr, G. A. Hamilton, L. A. McPartlin, S. Chung, K.-Y. Suh, D. E. Ingber, Human kidney proximal tubule-on-a-chip for drug transport and nephrotoxicity assessment. Integrative Biology 5, 1119-1129 (2013).
15. K. A. Homan, D. B. Kolesky, M. A. Skylar-Scott, J. Herrmann, H. Obuobi, A. Moisan, J. A. Lewis, Bioprinting of 3D convoluted renal proximal tubules on perfusable chips. Scientific reports 6, 1-13 (2016).
16. S. Musah, A. Mammoto, T. C. Ferrante, S. S. Jeanty, M. Hirano-Kobayashi, T. Mammoto, K. Roberts, S. Chung, R. Novak, M. Ingram, F.-D. Tohid, K. Sandeep, J. C. Weaver, G. M. Church, D. E. Ingber, Mature induced-pluripotent-stem-cell-derived human podocytes reconstitute kidney glomerular-capillary-wall function on a chip. Nature biomedical engineering 1, 1-12 (2017).
17. J. H. Low, P. Li, E. G. Y. Chew, B. Zhou, K. Suzuki, T. Zhang, M. M. Lian, M. Liu, E. Aizawa, C. R. Esteban, K. S. M. Yong, Q. Chen, J. M. Campistol, M. Fang, C. C. Khor, J. N. Foo, J. C. Izpisua Belmonte, Y. Xia, Generation of human PSC-derived kidney organoids with patterned nephron segments and a de novo vascular network. Cell Stem Cell 25, 373-387. e379 (2019).
18. O. Zaika, M. Mamenko, J. Berrout, N. Boukelmoune, R. G. O'Neil, O. Pochynyuk, TRPV4 dysfunction promotes renal cystogenesis in autosomal recessive polycystic kidney disease. Journal of the American Society of Nephrology 24, 604-616 (2013).
19. K. A. Homan, N. Gupta, K. T. Kroll, D. B. Kolesky, M. Skylar-Scott, T. Miyoshi, D. Mau, M. T. Valerius, T. Ferrante, J. V. Bonventre, J. A. Lewis, R. Morizane, Flow-enhanced vascularization and maturation of kidney organoids in vitro. Nature methods 16, 255-262 (2019).
20. V. E. Torres, P. C. Harris, Strategies targeting cAMP signaling in the treatment of polycystic kidney disease. Journal of the American Society of Nephrology 25, 18-32 (2014).
21. D.-C. Fischer, U. Jacoby, L. Pape, C. J. Ward, E. Kuwertz-Broeking, C. Renken, H. Nizze, U. Querfeld, B. Rudolph, D. E. Mueller-Wiefel, C. Bergmann, D. Haffner, Activation of the AKT/mTOR pathway in autosomal recessive polycystic kidney disease (ARPKD). Nephrology Dialysis Transplantation 24, 1819-1827 (2009).
22. K. M. Dell, The role of cilia in the pathogenesis of cystic kidney disease. Current opinion in pediatrics 27, 212 (2015).
23. F. Lina, L. M. Satlinb, Polycystic kidney disease: the cilium as a common pathway in cystogenesis. Current opinion in pediatrics 16, 171-176 (2004).
24. M. Ma, X. Tian, P. Igarashi, G. J. Pazour, S. Somlo, Loss of cilia suppresses cyst growth in genetic models of autosomal dominant polycystic kidney disease. Nature genetics 45, 1004-1012 (2013).
25. L. Shao, W. El-Jouni, F. Kong, J. Ramesh, R. S. Kumar, X. Shen, J. Ren, S. Devendra, A. Dorschel, M. Wu, I. Barrera, A. Tabari, K. Hu, N. Haque, I. Yambayev, S. Li, A. Kumar, T. R. Behera, G. McDonough, M. Furuichi, M. Xifaras, T. Lu, R. M. Alhayaza, K. Miyabayashi, Q. Fan, A. K. Ajay, J. Zhou, Genetic reduction of cilium length by targeting intraflagellar transport 88 protein impedes kidney and liver cyst formation in mouse models of autosomal polycystic kidney disease. Kidney international 98, 1225-1241 (2020).
26. A. R. Gallagher, S. Somlo, Loss of cilia does not slow liver disease progression in mouse models of autosomal recessive polycystic kidney disease. Kidney360 1, 962 (2020).
27. V. E. Torres, P. C. Harris, Mechanisms of disease: autosomal dominant and recessive polycystic kidney diseases. Nature clinical practice Nephrology 2, 40-55 (2006).
28. F. Martino, A. R. Perestrelo, V. Vinarsky, S. Pagliari, G. Forte, Cellular mechanotransduction: from tension to function. Frontiers in physiology 9, 824 (2018).
29. D. E. Discher, P. Janmey, Y.-l. Wang, Tissue cells feel and respond to the stiffness of their substrate. Science 310, 1139-1143 (2005).
30. K. Kaarj, J.-Y. Yoon, Methods of delivering mechanical stimuli to organ-on-a-chip. Micromachines 10, 700 (2019).
31. A. Colom, E. Derivery, S. Soleimanpour, C. Tomba, M. Dal Molin, N. Sakai, M. González-Gaitán, S. Matile, A. Roux, A fluorescent membrane tension probe. Nature chemistry 10, 1118-1125 (2018).
32. V. Raghavan, Y. Rbaibi, N. M. Pastor-Soler, M. D. Carattino, O. A. Weisz, Shear stress-dependent regulation of apical endocytosis in renal proximal tubule cells mediated by primary cilia. Proceedings of the National Academy of Sciences 111, 8506-8511 (2014).
33. W. Liu, S. Xu, C. Woda, P. Kim, S. Weinbaum, L. M. Satlin, Effect of flow and stretch on the [Ca2+] i response of principal and intercalated cells in cortical collecting duct. American Journal of Physiology-Renal Physiology 285, F998-F1012 (2003).
34. T. Malekshahabi, N. Khoshdel Rad, A. L. Serra, R. Moghadasali, Autosomal dominant polycystic kidney disease: Disrupted pathways and potential therapeutic interventions. Journal of cellular physiology 234, 12451-12470 (2019).
35. F. A. Belibi, C. L. Edelstein, Novel targets for the treatment of autosomal dominant polycystic kidney disease. Expert opinion on investigational drugs 19, 315-328 (2010).
36. T. B. Malas, C. Formica, W. N. Leonhard, P. Rao, Z. Granchi, M. Roos, D. J. Peters, P. A. 't Hoen, Meta-analysis of polycystic kidney disease expression profiles defines strong involvement of injury repair processes. American Journal of Physiology-Renal Physiology 312, F806-F817 (2017).
37. Y. M. Woo, J.-B. Bae, Y.-H. Oh, Y.-G. Lee, M. J. Lee, E. Y. Park, J.-K. Choi, S. Lee, Y. Shin, J. Lyu, H.-Y. Jung, Y.-S. Lee, Y.-H. Hwang, Y.-J. Kim, J. H. Park, Genome-wide methylation profiling of ADPKD identified epigenetically regulated genes associated with renal cyst development. Human genetics 133, 281-297 (2014).
38. K. J. Curtis, T. R. Coughlin, D. E. Mason, J. D. Boerckel, G. L. Niebur, Bone marrow mechanotransduction in porcine explants alters kinase activation and enhances trabecular bone formation in the absence of osteocyte signaling. Bone 107, 78-87 (2018).
39. J. Kroon, N. Heemskerk, M. J. Kalsbeek, V. de Waard, J. van Rijssel, J. D. van Buul, Flow-induced endothelial cell alignment requires the RhoGEF Trio as a scaffold protein to polarize active Rac1 distribution. Molecular biology of the cell 28, 1745-1753 (2017).
40. C. Smith, Drug target validation: Hitting the target. Nature 422, 342-345 (2003).
41. P. Maxwell, HIF-1: an oxygen response system with special relevance to the kidney. Journal of the American Society of Nephrology 14, 2712-2722 (2003).
42. A. N. Mahmoodi, P. Y. Kim, Ketorolac. StatPearls [Internet], (2021).
43. J. C. Brutzkus, M. Shahrokhi, M. Varacallo, Naproxen. StatPearls [Internet], (2021).
44. Y. Aikawa, K. Morimoto, T. Yamamoto, H. Chaki, A. Hashiramoto, H. Narita, S. Hirono, S. Shiozawa, Treatment of arthritis with a selective inhibitor of c-Fos/activator protein-1. Nature biotechnology 26, 817-823 (2008).
45. H. Makino, S. Seki, Y. Yahara, S. Shiozawa, Y. Aikawa, H. Motomura, M. Nogami, K. Watanabe, T. Sainoh, H. Ito, N. Tsumaki, Y. Kawaguchi, M. Yamazaki, T. Kimura, A selective inhibition of c-Fos/activator protein-1 as a potential therapeutic target for intervertebral disc degeneration and associated pain. Scientific reports 7, 1-14 (2017).
46. T. I. Oprea, L. A. Sklar, J. O. Agola, Y. Guo, M. Silberberg, J. Roxby, A. Vestling, E. Romero, Z. Surviladze, C. Murray-Krezan, A. Waller, O. Ursu, L. G. Hudson, A. Wandinger-Ness, Novel activities of select NSAID R-enantiomers against Rac1 and Cdc42 GTPases. PloS one 10, e0142182 (2015).
47. K. C. Duggan, M. J. Walters, J. Musee, J. M. Harp, J. R. Kiefer, J. A. Oates, L. J. Marnett, Molecular basis for cyclooxygenase inhibition by the non-steroidal anti-inflammatory drug naproxen. Journal of biological chemistry 285, 34950-34959 (2010).
48. J. D. Buenrostro, B. Wu, H. Y. Chang, W. J. Greenleaf, ATAC-seq: a method for assaying chromatin accessibility genome-wide. Current protocols in molecular biology 109, 21.29. 21-21.29. 29 (2015).
49. Y. Muto, P. C. Wilson, N. Ledru, H. Wu, H. Dimke, S. S. Waikar, B. D. Humphreys, Single cell transcriptional and chromatin accessibility profiling redefine cellular heterogeneity in the adult human kidney. Nature communications 12, 1-17 (2021).
50. H. Wu, Y. Kirita, E. L. Donnelly, B. D. Humphreys, Advantages of single-nucleus over single-cell RNA sequencing of adult kidney: rare cell types and novel cell states revealed in fibrosis. Journal of the American Society of Nephrology 30, 23-32 (2019).
51. J. H. Moyer, M. J. Lee-Tischler, H.-Y. Kwon, J. J. Schrick, E. D. Avner, W. E. Sweeney, V. L. Godfrey, N. L. Cacheiro, J. E. Wilkinson, R. P. Woychik, Candidate gene associated with a mutation causing recessive polycystic kidney disease in mice. Science 264, 1329-1333 (1994).
52. J. Fitzgerald, M. Hughes-Fulford, Mechanically induced c-fos expression is mediated by cAMP in MC3T3-E1 osteoblasts. The FASEB journal 13, 553-557 (1999).
53. L. J. Sundberg, L. M. Galante, H. M. Bill, C. P. Mack, J. M. Taylor, An endogenous inhibitor of focal adhesion kinase blocks Rac1/JNK but not Ras/ERK-dependent signaling in vascular smooth muscle cells. Journal of Biological Chemistry 278, 29783-29791 (2003).
54. K. Okumura, A. Itoh, E. Isogai, K. Hirose, Y. Hosokawa, Y. Abiko, T. Shibata, M. Hirata, H. Isogai, C-terminal domain of human CAP18 antimicrobial peptide induces apoptosis in oral squamous cell carcinoma SAS-H1 cells. Cancer letters 212, 185-194 (2004).
55. D. Nettersheim, S. Jostes, M. Fabry, F. Honecker, V. Schumacher, J. Kirfel, G. Kristiansen, H. Schorle, A signaling cascade including ARID1A, GADD45B and DUSP1 induces apoptosis and affects the cell cycle of germ cell cancers after romidepsin treatment. Oncotarget 7, 74931 (2016).
56. L. Zhang, J. Gao, L. Li, Z. Li, Y. Du, Y. Gong, The neuronal pentraxin II gene (NPTX2) inhibit proliferation and invasion of pancreatic cancer cells in vitro. Molecular biology reports 38, 4903-4911 (2011).
57. J. Ma, K. Nie, D. Redmond, Y. Liu, O. Elemento, D. Knowles, W. Tam, EBV-miR-BHRF1-2 targets PRDM1/Blimp1: potential role in EBV lymphomagenesis. Leukemia 30, 594-604 (2016).
58. F. Guo, Z. Xu, Y. Zhang, P. Jiang, G. Huang, S. Chen, X. Lyu, P. Zheng, X. Zhao, Y. Zeng, S. Wang, F. He, FXR induces SOCS3 and suppresses hepatocellular carcinoma. Oncotarget 6, 34606-34616 (2015).
59. G. Den Hartog, R. Chattopadhyay, A. Ablack, E. H. Hall, L. D. Butcher, A. Bhattacharyya, L. Eckmann, P. R. Harris, S. Das, P. B. Ernst, S. E. Crowe, Regulation of Rac1 and Reactive Oxygen Species Production in Response to Infection of Gastrointestinal Epithelia. PLOS Pathogens 12, e1005382 (2016).
60. T.-S. Jou, W. James Nelson, Effects of regulated expression of mutant RhoA and Rac1 small GTPases on the development of epithelial (MDCK) cell polarity. The Journal of cell biology 142, 85-100 (1998).
61. G. Yao, X. Su, V. Nguyen, K. Roberts, X. Li, A. Takakura, M. Plomann, J. Zhou, Polycystin-1 regulates actin cytoskeleton organization and directional cell migration through a novel PC1-Pacsin 2-N-Wasp complex. Human molecular genetics 23, 2769-2779 (2014).
62. Y. M. Woo, D. Y. Kim, N. J. Koo, Y.-M. Kim, S. Lee, J. Y. Ko, Y. Shin, B. H. Kim, H. Mun, S. Choi, E. J. Lee, J.-O. Shin, E. Y. Park, J. Bok, J. H. Park, Profiling of miRNAs and target genes related to cystogenesis in ADPKD mouse models. Scientific Reports 7, 1-12 (2017).
63. L. Locatelli, J. A. Maier, Cytoskeletal remodeling mimics endothelial response to microgravity. Frontiers in Cell and Developmental Biology 9, (2021).
64. Y. Qiu, A. C. Brown, D. R. Myers, Y. Sakurai, R. G. Mannino, R. Tran, B. Ahn, E. T. Hardy, M. F. Kee, S. Kumar, G. Bao, T. H. Barker, W. A. Lam, Platelet mechanosensing of substrate stiffness during clot formation mediates adhesion, spreading, and activation. Proceedings of the National Academy of Sciences of the United States of America 111, 14430-14435 (2014).
65. X. Huang, Y. Shen, Y. Zhang, L. Wei, Y. Lai, J. Wu, X. Liu, X. Liu, Rac1 mediates laminar shear stress-induced vascular endothelial cell migration. Cell adhesion & migration 7, 472-478 (2013).
66. H. Kahsay, Assessment and treatment of pain in pediatric patients. Current Pediatric Research, (2017).
67. J. Zhang, Z. Hou, C. Yao, Y. Yu, Purification and properties of lipase from a Bacillus strain for catalytic resolution of (R)-Naproxen. Journal of Molecular Catalysis B: Enzymatic 18, 205-210 (2002).
68. B. S. Freedman, A. Q. Lam, J. L. Sundsbak, R. Iatrino, X. Su, S. J. Koon, M. Wu, L. Daheron, P. C. Harris, J. Zhou, J. V. Bonventre, Reduced ciliary polycystin-2 in induced pluripotent stem cells from polycystic kidney disease patients with PKD1 mutations. Journal of the American Society of Nephrology 24, 1571-1586 (2013).
69. R. Morizane, J. V. Bonventre, Generation of nephron progenitor cells and kidney organoids from human pluripotent stem cells. Nature protocols 12, 195-207 (2017).
70. N. Gupta, K. Susa, Y. Yoda, J. V. Bonventre, M. T. Valerius, R. Morizane, CRISPR/Cas9-based Targeted Genome Editing for the Development of Monogenic Diseases Models with Human Pluripotent Stem Cells. Current protocols in stem cell biology 45, e50 (2018).
71. D. B. Kolesky, K. A. Homan, M. A. Skylar-Scott, J. A. Lewis, Three-dimensional bioprinting of thick vascularized tissues. Proceedings of the national academy of sciences 113, 3179-3184 (2016).
72. R. G. Morgan, A. C. Chambers, D. N. Legge, S. Coles, A. Greenhough, A. C. Williams, Optimized delivery of siRNA into 3D tumor spheroid cultures in situ. Scientific reports 8, 1-10 (2018).
73. K. Kuželová, D. Grebeňová, Z. Hrkal, Labeling of apoptotic JURL-MK1 cells by fluorescent caspase-3 inhibitor FAM-DEVD-fmk occurs mainly at site (s) different from caspase-3 active site. Cytometry Part A: The Journal of the International Society for Analytical Cytology 71, 605-611 (2007).
74. J. J. Alvarado-Sansininea, L. Sánchez-Sánchez, H. López-Muñoz, M. L. Escobar, F. Flores-Guzmán, R. Tavera-Hernández, M. Jimenez-Estrada, Quercetagetin and patuletin: Antiproliferative, necrotic and apoptotic activity in tumor cell lines. Molecules 23, 2579 (2018).
75. K. Zhou, J. Rao, Z.-h. Zhou, X.-h. Yao, F. Wu, J. Yang, L. Yang, X. Zhang, Y.-h. Cui, X.-W. Bian, Y. Shi, Y.-f. Ping, RAC1-GTP promotes epithelial-mesenchymal transition and invasion of colorectal cancer by activation of STAT3. Laboratory Investigation 98, 989-998 (2018).
76. G. Xiao, X. Wang, J. Wang, L. Zu, G. Cheng, M. Hao, X. Sun, Y. Xue, J. Lu, J. Wang, CXCL16/CXCR6 chemokine signaling mediates breast cancer progression by pERK1/2-dependent mechanisms. Oncotarget 6, 14165 (2015).
77. M.-G. Ryou, G. R. Choudhury, W. Li, A. Winters, F. Yuan, R. Liu, S.-H. Yang, Methylene blue-induced neuronal protective mechanism against hypoxia-reoxygenation stress. Neuroscience 301, 193-203 (2015).
78. K. Hiratsuka, T. Monkawa, T. Akiyama, Y. Nakatake, M. Oda, S. K. Goparaju, H. Kimura, N. Chikazawa-Nohtomi, S. Sato, K. Ishiguro, S. Yamaguchi, S. Suzuki, R. Morizane, S. B. H. Ko, H. Itoh, M. S. H. Ko, Induction of human pluripotent stem cells into kidney tissues by synthetic mRNAs encoding transcription factors. Scientific reports 9, 1-13 (2019).
79. H. B. Pearson, J. Li, V. S. Meniel, C. M. Fennell, P. Waring, K. G. Montgomery, R. J. Rebello, A. A. Macpherson, S. Koushyar, L. Furic, C. Cullinane, R. W. Clarkson, M. J. Smalley, K. J. Simpson, T. J. Phesse, P. R. Shepherd, P. O. Humbert, O. J. Sansom, W. A. Phillips, Identification of Pik3ca mutation as a genetic driver of prostate cancer that cooperates with Pten loss to accelerate progression and castration-resistant growth. Cancer Discovery 8, 764-779 (2018).
80. Crosas-Molist E, Samain R, Kohlhammer L, Orgaz J L, George S L, Maiques O, Barcelo J, Sanz-Moreno V. Rho GTPase signaling in cancer progression and dissemination. Physiol Rev. 2022; 102(1):455-510. Epub 20210920. doi: 10.1152/physrev.00045.2020. PubMed PMID: 34541899.
81. Bagci H, Sriskandarajah N, Robert A, Boulais J, Elkholi I E, Tran V, Lin Z Y, Thibault M P, Dube N, Faubert D, Hipfner D R, Gingras A C, Cote J F. Mapping the proximity interaction network of the Rho-family GTPases reveals signalling pathways and regulatory mechanisms. Nature cell biology. 2020; 22(1):120-34. Epub 20191223. doi: 10.1038/s41556-019-0438-7. PubMed PMID: 31871319.
82. PCT/US2018/036677

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating autosomal recessive polycystic kidney disease (ARPKD), the method comprising administering a therapeutically effective amount of an inhibitor of Rac family small GTPase 1 (RAC1) and/or an inhibitor of Fos proto-oncogene, AP-1 transcription factor subunit (FOS) to a subject in need thereof.

2. The method of claim 1, wherein the subject has been diagnosed with ARPKD.

3. The method of claim 2, wherein the subject has a mutation in PKHD1 or has been confirmed to have a mutation in PKHD1.

4. The method of claim 1, wherein the treatment results in a reduction in cyst formation or growth and/or improved renal function

5. The method of claim 4, wherein improved renal function is determined by increased glomerular filtration rate (GFR), reduced serum creatinine level, and/or reduced Blood urea nitrogen (BUN).

6. The method of claim 1, wherein the inhibitor of FOS is a benzophenone derivative comprising the formula 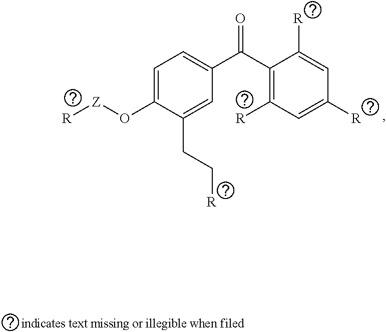
wherein
R1 represents, for example, an optionally substituted heterocyclic group, or a substituted phenyl group; Z represents, for example, an alkylene group;
R2 represents, for example, a carboxyl group optionally protected with alkyl;
R3 represents, for example, an optionally protected hydroxyl group;
R4 represents, for example, an optionally substituted cycloalkyloxy group; and
R5 represents, for example, a hydrogen atom,
or a pharmaceutically acceptable salt thereof.

7. The method of claim 6, wherein the inhibitor of FOS is T-5224 (3-{5-[4-(cyclopentyloxy)-2-hydroxybenzoyl]-2-[(3-hydroxy-1,2-benzisoxazol-6-yl)methoxy]phenyl}propionic acid).

8. The method of claim 1, wherein the inhibitor of RAC1 is an NSAID; ZINC69391 or analog 1A-116; 1D-142; anthraquinone-quinazoline hybrid 7B; NSC23766; a thioquinoline derivative such as EHT 1864; GYS32661; or MBQ-167.

9. The method of claim 8, wherein the NSAID is R-Naproxen or R-Ketorolac.

10. The method of claim 1, wherein the inhibitor of RAC1 is an inhibitory nucleic acid targeting RAC1, and/or the inhibitor of FOS is an inhibitory nucleic acid targeting FOS.

11. The method of claim 10, wherein the inhibitory nucleic acid targeting RAC1 or the inhibitory nucleic acid targeting FOS is an antisense RNA, antisense DNA, mixmer, gapmer, antisense oligonucleotides comprising one or more modified linkages, interference RNA (RNAi), short interfering RNA (siRNA), or a short, hairpin RNA (shRNA).

12.-22. (canceled)


SEQUENCE LISTING (13 sequences)

SEQ ID NO: 1 - DNA, 20 bp, synthetic construct
  source 1..20: mol_type = other DNA
tcatttttgatggtaggttg

SEQ ID NO: 2 - DNA, 20 bp, synthetic construct
  source 1..20: mol_type = other DNA
gcggcggaaaatagcctttg

SEQ ID NO: 3 - DNA, 21 bp, synthetic construct
  source 1..21: mol_type = other DNA
gatcacacgttccacctcatc

SEQ ID NO: 4 - DNA, 23 bp, synthetic construct
  source 1..23: mol_type = other DNA
ggccattgacttcatagactcca

SEQ ID NO: 5 - DNA, 20 bp, synthetic construct
  source 1..20: mol_type = other DNA
aactcaaaggcctcgtccag

SEQ ID NO: 6 - DNA, 19 bp, synthetic construct
  source 1..19: mol_type = other DNA
acgggcaaggacactatgg

SEQ ID NO: 7 - DNA, 21 bp, synthetic construct
  source 1..21: mol_type = other DNA
attggacacgtttgctctgag

SEQ ID NO: 8 - DNA, 21 bp, synthetic construct
  source 1..21: mol_type = other DNA
aagcaactggatgcgctatgt

SEQ ID NO: 9 - DNA, 23 bp, synthetic construct
  source 1..23: mol_type = other DNA
gggatgggcttaatggtgtagaa

SEQ ID NO: 10 - DNA, 22 bp, synthetic construct
  source 1..22: mol_type = other DNA
atcagaacgatttggtcgaggc

SEQ ID NO: 11 - DNA, 21 bp, synthetic construct
  source 1..21: mol_type = other DNA
atacttgcgcttcgtctcacg

SEQ ID NO: 12 - DNA, 21 bp, synthetic construct
  source 1..21: mol_type = other DNA
gacgtatgcagccaaaggcta

SEQ ID NO: 13 - DNA, 21 bp, synthetic construct
  source 1..21: mol_type = other DNA
gcagctcgttgtgaaaagact